(19) United States
(12) Patent Application Publication
(10) Pub. No.: US 2007/0269824 A1
(43) Pub. Date: Nov. 22, 2007

(54) Methods and systems for evaluating health risk factors by measurement of DNA damage and DNA repair

(76) Inventors: Jeffrey Albrecht, Santa Monica, CA (US); Andrew Conrad, Malibu, CA (US); Francoise Gala, West Hollywood, CA (US); Russell Grant, Thousand Oaks, CA (US)

Correspondence Address: KILPATRICK STOCKTON LLP, 1001 WEST FOURTH STREET, WINSTON-SALEM, NC, 27101, US

(21) Appl. No.: 11/713,451

(22) Filed: Mar. 2, 2007

Related U.S. Application Data
(60) Provisional application No. 60/778,284, filed on Mar. 2, 2006.

(51) Int. Cl. C12Q 1/68

(52) U.S. Cl. 435/6

(57) ABSTRACT

Disclosed are methods for measuring the effects of environmental, physiological, or lifestyle variables on DNA damage and DNA repair activity as well as the use of measurements of DNA damage and DNA repair activity to predict increased risk for disease. Embodiments of the methods involve the use of a combination of assays to measure DNA damage and DNA repair activity in an individual and comparing these measurements to suitable controls using the selected assays for normal healthy individuals of varying ages. In other embodiments, the methods may comprise a comparison of DNA damage levels to DNA repair levels to obtain an apparent net measurement of DNA damage accumulation.

### CROSS REFERENCE TO RELATED APPLICATIONS

The present application claims priority under 35 U.S.C. § 119(e) from U.S. Provisional Patent Application Ser. No. 60/778,284, filed Mar. 2, 2006. The disclosure of U.S. Provisional Patent Application 60/778,284 is hereby incorporated by reference in its entirety herein.

### FIELD OF THE INVENTION

The invention relates to evaluating health risk factors by measurement of DNA damage and DNA repair.

### BACKGROUND OF THE INVENTION

Cellular DNA may be damaged by both endogenous and environmental factors. For example, production of ATP within cells via oxidative phosphorylation can result in reactive oxygen species, or free radicals, which can alter and damage proteins, lipids and DNA via oxidation. Oxidation of DNA bases (guanine preferentially) can lead to mutations. Environmental agents such as ultraviolet radiation, x-rays, gamma rays, mutagenic compounds such as hydrocarbons, and cancer chemotherapy and radiotherapy may also contribute to DNA damage. Because DNA damage can interfere with the integrity and accessibility of information encoded in the genome, a number of DNA repair mechanisms have evolved to rapidly correct such damage as it occurs.

The balance between DNA damage and DNA repair activity may be vital to normal cellular functioning and to an individual's longevity and health. When the rate of ongoing DNA damage outpaces the rate of DNA repair, DNA damage may accumulate. This accumulation can cause cells to undergo apoptosis, become cancerous, or may contribute to the development of various diseases and the acceleration of the aging process.

To date, some studies have recognized the relationship between the health of a particular tissue and DNA damage and/or DNA repair. For example, investigators have studied the markers 8-hydroxydeoxyguanosine and the free base 8-oxoguanine (8-oxo-G) as markers of DNA damage, repair, and oxidative stress. Oxidative DNA damage has been shown to be higher in uterine myomas, in patients with bone metastasis from breast, colon, and prostate cancer, and in the plasma, urine and cerebrospinal fluid of patients with amyotrophic lateral sclerosis (Foksinski et al. (2000) Free Radical Biology & Medicine 29:597-601; Rozalski et al. (2002) Cancer Epidemiology, Biomarkers, and Prevention 11: 1072-1075; Bogdanov et al. (2000) Free Radical Biology & Medicine 29:652-658). The levels of such markers has also been suggested to be linked to a combination of lifestyle, environmental, and genetic factors (Phillips et al. (1988) Nature 336:790-792; Loft et al. (1992) Carcinogenesis 13:2241-2247; Kiyosawa et al. (1990) Free Radical Res. Comm. 11:23-27; Asami et al. (1997) Carcinogenesis 18:1763-6; Asami et al. (1996) Cancer Res. 56:2546-2549). Such lifestyle and genetic factors include antioxidant supplementation (Halliwell (1996) Free Radic. Res. 25:57-74); smoking, gender and body mass index (Loft et al. (1992) Carcinogenesis 13:2241-2247); diet rich in fruits and vegetables (Halliwell (2002) Free Radic. Biol. Med. 32:968-974); exercise (Poulsen et al. (1999) Proc. Nutr. Soc. 58:1007-1014); exercise, working conditions, meat intake, body mass index, and smoking (Kasai et al. (2001) Jpn. J. Cancer Res. 92:9-15); gender (Proteggente (2002) Free Radic. Res. 36:157-162); and smoking, body composition, calorie restriction, and age (Loft et al. (1993) J. Toxicol. Environ. Health 40:391-404).

In spite of the recognized relationship between DNA damage and disease, assays to detect DNA damage and repair activity are not widely used by clinicians as part of overall health promotion or disease detection and prevention. Furthermore, although studies involving individual tests for DNA damage and repair activity have been conducted by various groups, methods involving a more comprehensive battery of tests for measuring both DNA damage induction and DNA repair have not been used. For these reasons, methods for monitoring DNA damage and repair activity that provide a more comprehensive and meaningful individualized profile of disease risk or the impact of various environmental, physiological, or lifestyle variables on overall health are needed.

### SUMMARY OF THE INVENTION

Embodiments of the present invention comprise methods and systems for evaluating health risk factors by measurement of DNA damage and DNA repair. The present invention may be embodied in a variety of ways.

In an embodiment, the present invention may comprise a method to correlate effect of at least one variable on at least one of DNA damage or DNA repair in an individual. For example, in certain embodiments, the method may comprise the step of measuring DNA damage and DNA repair in the individual. In some embodiments, the method may comprise using a plurality of assays to measure DNA damage and/or a plurality of assays to measure DNA repair. The method may further comprise determining whether the amount of at least one of DNA damage or DNA repair as measured in the individual differs from the amount of at least one of DNA damage or DNA repair for a plurality of control samples, or reference ranges derived from a plurality of controls. The method may also comprise determining if there is a correlation between the levels of at least one of DNA damage or DNA repair in the individual and the variable of interest.

There are additional features of the invention which will be described hereinafter. It is to be understood that the invention is not limited in its application to the details set forth in the following claims, description and figures. The invention is capable of other embodiments and of being practiced or carried out in various ways.

### BRIEF DESCRIPTION OF THE DRAWINGS

Various features, aspects and advantages of the present invention will become more apparent with reference to the following figures.

FIG. 1 shows a schematic representation of methods for evaluating health risk factors by measurement of DNA damage and DNA repair, wherein panel A shows embodiments of methods to correlate the effect of a lifestyle, environmental or physiological variable with DNA damage and/or repair, and panel B shows embodiments of methods to correlate disease risk with DNA damage and/or repair in accordance with alternate embodiments of the present invention.

FIG. 2 shows a system for evaluating health risk factors by measurement of DNA damage and DNA repair in accordance with an embodiment of the present invention.

FIG. 3 shows a kit for evaluating health risk factors by measurement of DNA damage and DNA repair in accordance with an embodiment of the present invention.

FIG. 4 shows representational graphs of Real-Time PCR amplification of DNA repair enzymes (DREs) for use in DRE assays in accordance with alternate embodiments of the present invention.

FIG. 5 shows a schematic diagram depicting the quantification of DREs against a normalization factor, an internal control housekeeping gene, Ubiquitin C (UBC) in accordance with alternate embodiments of the present invention.

FIG. 6 shows a schematic diagram depicting methods and steps for a DNA Repair Capacity Analysis assay in accordance with alternate embodiments of the present invention.

FIG. 7 provides a description of the DNA Damage Susceptibility Assay in accordance with alternate embodiments of the present invention.

### DETAILED DESCRIPTION

For the purposes of this specification, unless otherwise indicated, all numbers expressing quantities of ingredients, reaction conditions, and so forth used in the specification are to be understood as being modified in all instances by the term “about.” Accordingly, unless indicated to the contrary, the numerical parameters set forth in the following specification are approximations that can vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g. 1 to 6.1, and ending with a maximum value of 10 or less, e.g., 5.5 to 10. Additionally, any reference referred to as being “incorporated herein” is to be understood as being incorporated in its entirety.

It is further noted that, as used in this specification, the singular forms “a,” “an,” and “the” include plural referents unless expressly and unequivocally limited to one referent. The term “or” is used interchangeably with the term “and/or” unless the context clearly indicates otherwise.

Unless defined otherwise, all technical and scientific terms used herein have the same meaning as commonly understood by one of ordinary skill in the art. Practitioners are particularly directed to Current Protocols in Molecular Biology (see e.g. Ausubel, F. M. et al., Short Protocols in Molecular Biology, 4^{th} Ed., Chapter 2, John Wiley & Sons, N.Y.) for definitions and terms of the art. Abbreviations for amino acid residues are the standard 3-letter and/or 1-letter codes used in the art to refer to one of the 20 common L-amino acids.

A “nucleic acid” is a polynucleotide such as deoxyribonucleic acid (DNA) or ribonucleic acid (RNA). The term is used to include single-stranded nucleic acids, double-stranded nucleic acids, and RNA and DNA made from nucleotide or nucleoside analogues.

“Polypeptide” and “protein” are used interchangeably herein to describe protein molecules that may comprise either partial or full-length proteins. As is known in the art, “proteins”, “peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. Typically, the amino acids making up a protein are numbered in order, starting at the amino terminal residue and increasing in the direction toward the carboxy terminal residue of the protein.

As is known in the art, conditions for hybridizing or annealing nucleic acid sequences to each other can be described as ranging from low to high stringency. Generally, highly stringent hybridization conditions refer to washing hybrids in low salt buffer at high temperatures. Hybridization may be to filter bound DNA using hybridization solutions standard in the art such as 0.5M NaHPO₄, 7% sodium dodecyl sulfate (SDS), at 65° C., and washing in 0.25 M NaHPO₄, 3.5% SDS followed by washing 0.1×SSC/0.1% SDS at a temperature ranging from room temperature to 68° C. depending on the length of the probe. For example, a high stringency wash comprises washing in 6×SSC/0.05% sodium pyrophosphate at 37° C. for a 14 base oligonucleotide probe, or at 48° C. for a 17 base oligonucleotide probe, or at 55° C. for a 20 base oligonucleotide probe, or at 60° C. for a 25 base oligonucleotide probe, or at 65° C. for a nucleotide probe about 250 nucleotides in length. Nucleic acid probes may be labeled with radionucleotides by end-labeling with, for example, [γ-³²P]ATP, or incorporation of radiolabeled nucleotides such as [α-³²P]dCTP by random primer labeling. Alternatively, probes may be labeled by incorporation of nucleotides that are labeled with biotin, fluorescein or digoxygenin labeled nucleotides, and the probe detected using Streptavidin, anti-fluorescein antibodies, or anti-digoxygenin antibodies, respectively.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981, Adv. Appl. Math. 2:482; Needleman and Wunsch, 1970, J. Mol. Biol. 48:443; Pearson and Lipman, 1988, Proc. Natl. Acad. Sci., USA, 85:2444) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, the percent identity of two sequences may be determined using GCG with a gap weight of 1, such that each amino acid gap is weighted as if it were a single amino acid mismatch between the two sequences.

As used herein, the term at least 90% identical thereto includes sequences that range from 90 to 99.99% identity to the indicated sequences and includes all ranges in between. Thus, the term at least 90% identical thereto includes sequences that are 91, 91.5, 92, 92.5, 93, 93.5. 94, 94.5, 95, 95.5, 96, 96.5, 97, 97.5, 98, 98.5, 99, 99.5 percent identical to the indicated sequence. Similarly the term “at least 70% identical includes sequences that range from 70 to 99.99% identical, with all ranges in between. The determination of percent identity is determined using the algorithms described here.

The Polymerase Chain Reaction (PCR) takes advantage of the self-replicating nature of DNA to provide for the replicaton of DNA molecules in vitro. In PCR, double stranded DNA is heated to a temperature where the strands separate. Then, primers (oligonucleotide sequences that are complimentary to the ends of the region to be amplified are allowed to hybridize (i.e., anneal) to the DNA template (i.e., the DNA to be replicated). Replication is initiated using a heat-stable DNA polymerase enzyme, e.g., a DNA polymerase isolated from Thermus Aquaticus (i.e., Taq DNA Polymerase). Use of this polymerase allows for cycling of the reaction through high temperatures required for denaturation (e.g, 95° C.) and mid-range temperatures (e.g., 65-72° C.) required for primer hybridization and elongation. Cycles of denaturation, annealing and polymerization are repeated such that the molecules replicate exponentially resulting in rapid and efficient amplification of the genetic material. The reaction can be carried out automatically with a thermocycler.

As used herein, DNA damage is a change in DNA structure that causes mispairing of the DNA. DNA damage may include, but is not be limited to, base oxidation (such as 8-hydroxyguanine and 8-hydroxyadenine); strand breaks; deamination; chemical modification; pyrimidine dimerization; crosslinking with proteins; interstrand crosslinks.

As used herein, DNA repair is the replacement of altered nucleotides or groups of nucleotides. This may be performed via base excision repair (BER) or nucleotide excision repair (NER). Briefly, BER involves removal of an incorrect or damaged base by an appropriate DNA N-glycosylase (“DNA Repair Enzyme”) to create an abasic site, with the sugar backbone of the strand intact. Nicking of that DNA strand by AP endonuclease (APE-1) upstream of the abasic site creates a 3′-OH terminus adjacent to the site. DNA polymerase replaces the correct nucleotide, using the opposite strand as a template and the remaining baseless “nucleotide” is removed by a DNA lyase. NER involves the recognition of damaged DNA strand regions based on their abnormal structure as well as on their abnormal chemistry, for example, in the case of bulky chemical modifications or crosslinking. Rather than individual nucleotides being replaced, short stretches are replaced. Briefly, after damage recognition, a multi-protein complex binds at the damaged site and double incisions are made on the damaged strand several nucleotides away from the site, on both the 5′ and 3′ sides. The damage-containing oligonucleotide is removed from between the two nicks. The resulting gap is filled in by a DNA polymerase and the 3′ end is attached to the remainder of the strand by a DNA ligase. Repair of damaged nucleotides prevents mispairing during replication, leading to potentially dangerous mutations in genes and therefore, health risks.

As used herein, a health risk factor is a factor that can effect the health of an individual and that may be related to changes in the individual's DNA.

As used herein, an “individual” may be any human or animal that is being tested using the methods of systems of the present invention. The individual may, in certain embodiments, be a mammal. Also, the individual may be a patient or a test subject.

As used herein, an environmental variable is a variable that originates in the individual's environment. A lifestyle variable is a variable that originates as a result of the individual's lifestyle. A physiological variable is a variable that originates as a result of the individual's physiology (e.g., genetic makeup). There may be overlap between variables, such that some variables may result from both environment and physiology (e.g., mutations induced by carcinogens), or from both lifestyle and environment (e.g., exposure to sunlight), or from both lifestyle and physiology (e.g., exposure to sunlight and the development of skin cancer). Such variables include but are not limited to, diet, physical activity, aging, pregnancy, stress, smoking, alcohol consumption, disease, disease treatment, drug treatment, antioxidant supplementation, cosmetic treatment (e.g., chemical peels or laser resurfacing), exposure to a carcinogen including toxic industrial chemicals, and exposure to X-ray and/or ultraviolet irradiation (e.g., tanning).

By “environmental, physiological, or lifestyle variable change” is intended a change in or exposure to variables that include, but are not limited to, diet, physical activity, aging, pregnancy, stress, smoking, alcohol consumption, disease, disease treatment, drug treatment, antioxidant supplementation, cosmetic treatment (e.g., chemical peels or laser resurfacing), exposure to a carcinogen including toxic industrial chemicals, and exposure to X-ray and/or ultraviolet irradiation (e.g., tanning).

As used herein, an adduct is an altered residue on a biological molecule such as an altered nucleotide on DNA. The alteration may be a binding event, such as oxidation of a double bond or crosslinking or it may be a loss, such as deamination. Also, as used herein, “DNA adduct assays” or “DNA adduct measurements” involve the measurement of the level of a given altered DNA base or nucleotide associated with DNA damage in various bodily fluids and tissues of an individual.

As used herein the term “reference ranges” refers to a range of test result values for a defined set of individuals of a relevant demographic group to which test sample results may be compared. The size and characteristics of the set of individuals and the relevant demographic group may vary from one test to another. In an embodiment, the reference ranges are based on observed results for a large number of individuals (for example, collected in clinical trials). Reference ranges may include range limits, generally a range of standard deviations from the average result. In an embodiment, the range limits are set at the average result +/−2 standard deviations.

Correlation of DNA Damage and/or DNA Repair to Disease Risk

Embodiments of the present invention may comprise methods and systems evaluating health risk factors by measuring DNA damage and/or DNA repair activity. The present invention may be embodied in a variety of ways.

For example, in some embodiments, the present invention may comprise methods or systems to correlate effect of a variable of interest to DNA damage and/or DNA repair in an individual. The variable of interest may be at least one of a lifestyle, environmental, or physiological variable. The variable of interest may be a variable such as diet, physical activity, aging, pregnancy, stress, smoking, alcohol consumption, disease, disease treatment, drug treatment, antioxidant supplementation, cosmetic treatment (e.g., chemical peels or laser resurfacing), exposure to a carcinogen including toxic industrial chemicals, and exposure to X-ray and/or ultraviolet irradiation (e.g., tanning).

Thus, the method of the present invention may, in certain embodiments, comprise measuring at least one of DNA damage and/or DNA repair in the individual. In some embodiments, the method may comprise a using a plurality of assays to measure DNA damage and/or a plurality of assays to measure DNA repair. Thus, the method may comprise performing a plurality of assays of DNA damage, and/or a plurality of assays of DNA repair, and/or at least one assay each of DNA damage and DNA repair. The method may further comprise determining whether the amount of at least one of DNA damage or DNA repair in the individual as measured by a particular assay or a plurality of assays differs from the amount of at least one of DNA damage or DNA repair for a plurality of control samples. In an embodiment, the control samples comprise reference ranges for DNA damage or DNA repair as derived from a plurality of controls. The method may also comprise determining if there is a correlation between the levels of at least one of DNA damage or DNA repair as measured by a particular assay or a plurality of assays in the individual to changes in the variable of interest.

For example, the method of the present invention may, in certain embodiments, comprise at least two measurements, either of DNA damage or DNA repair, or at least one of each DNA damage and DNA repair in the individual. The method may further comprise determining whether the results of the measurements of DNA damage and/or DNA repair as measured in the individual differ from the results of the same tests for a plurality of control samples. The values from control samples may comprise a range of values (i.e., a reference range) for each measurement. The method may also comprise determining if there is a correlation between the levels of at least two measurements of DNA damage and/or DNA repair in the individual to changes in the variable of interest.

In some embodiments, both DNA repair and DNA damage are measured. For example, in one embodiment, the method and systems may comprise obtaining measurements of both DNA damage and DNA repair activity in the individual, and determining whether levels of DNA damage and DNA repair in the individual are greater than or less than reference values (or a range of reference values) using the selected assays for normal healthy individuals. In an embodiment, the reference values (or the reference range) are from age-matched controls. If any abnormal results can be correlated with environmental, physiological, or lifestyle changes, the individual may have recently experienced or with a generally poor lifestyle (e.g., smoking, excessive alcohol intake, lack of exercise, poor diet), a change in DNA quality may, in certain embodiments, demonstrate a physical manifestation of the effect of such environmental, physiological, or lifestyle variables and serve to aid both individuals and their healthcare providers in making decisions about health maintenance.

In other embodiments, the present invention may comprise methods and systems for predicting increased risk for disease by measuring at least one of DNA damage and/or DNA repair indicators in an individual. In certain embodiments, the method for predicting disease risk may comprise the step of measuring both DNA damage and DNA repair in the individual. In some embodiments, the method may comprise a plurality of assays of DNA damage and/or a plurality of assays of DNA repair. Thus, the method may comprise performing a plurality of assays of DNA damage, and/or a plurality of assays of DNA repair, and/or at least one assay each of DNA damage and DNA repair. The method may further comprise determining whether the amount of at least one of DNA damage or DNA repair as measured in the individual differs from the amount of at least one of DNA damage or DNA repair for a plurality of control samples. The method may also comprise comparing the level of the at least one of DNA damage or DNA repair in the individual as measured by a particular assay or a plurality of assays to the control samples. In an embodiment, the method may also include determining if there is a correlation between a change in at least one of DNA damage or DNA repair and a change in risk of disease for the individual. In an embodiment, the control samples comprise a reference range (or a plurality of reference ranges) for DNA damage or DNA repair as derived from a plurality of controls. For example, in certain embodiments an increase in DNA damage or a decrease in DNA repair as measured by a particular assay or a plurality of assays are correlated to an increased risk of disease for the individual.

Thus, in some embodiments, the present invention may comprise methods and systems for predicting increased risk for disease by measuring at least two indicators of DNA damage or DNA repair or one of each in an individual. In certain embodiments, the method for predicting disease risk may comprise the step of measuring both DNA damage and DNA repair in the individual. The method may further comprise determining whether the amount of at least one measurement of DNA damage and one of DNA repair in the individual differ from the results of the same tests for a plurality of control samples. The method may also comprise comparing the level of at least two measurements of DNA damage or DNA repair or one of each in the individual to the control samples, wherein changes in the results of the same tests are correlated to a change in risk of disease for the individual. For example, in certain embodiments an increase in DNA damage or a decrease in DNA repair are correlated to an increased risk of disease for the individual.

For the methods and systems of the present invention, the control samples may comprise reference ranges for DNA damage or DNA repair as derived from a plurality of controls. In one embodiment, the controls may comprise a range of values (reference ranges) derived from healthy, age-matched individuals. Alternatively, the controls may comprise reference ranges derived from a plurality of test subjects. Or, the controls may comprise may be a baseline (e.g., time 0) sample for the individual being monitored. Or, other controls may be used, such as samples that provide information correlated to the health risk factor being assayed (e.g., samples from family members for a genetic disease; or samples from co-workers for an environmental variable).

For example, certain embodiments of the methods or systems of the present invention comprise obtaining measurements of both DNA damage and DNA repair activities in the individual, or a plurality of measurements of either DNA damage and/or DNA repair (e.g., a plurality of different assays specific for different indicators of DNA damage or DNA repair), determining whether levels of DNA damage and DNA repair in the individual are greater than or less than a range of values for age-matched healthy individuals using the same selected assays, and comparing the levels of both DNA damage and DNA repair.

In other embodiments, instead of using age-matched controls from healthy individuals to derive a range of control values, the methods of the present invention may comprise establishing baseline measurements of either DNA damage or DNA repair activity, or both DNA damage and DNA repair activity, or a plurality of measurements of either DNA damage and/or DNA repair, in an individual and then repeating the measurements of DNA damage and DNA repair activity using the same selected assays at one or more later time points. The method may further comprise comparing levels of both DNA damage and/or DNA repair at each time point and determining whether the apparent overall accumulation of DNA damage at later time points has increased or decreased compared to baseline measurements (or a range of values as provided by baseline measurements).

For example, in one embodiment, the present invention may comprise a method to correlate effect of at least one measurement of DNA damage or one measurement of DNA repair to an increased or a decreased risk for disease in an individual comprising the steps of: (a) using a plurality of assays to measure DNA damage and/or DNA repair in the individual at an initial time; (b) repeating the measurements of step (a) at one or more later time points; and (c) determining whether the amount of at least one measurement of DNA damage or DNA repair as measured for at least one later time point differs from the amount of at least one of DNA damage or DNA repair at the initial time point; and (d) determining if there is a correlation between a change in at least one of DNA damage or DNA repair and a change in the risk of the disease for the individual. In an embodiment, a change in at least one of DNA damage or DNA repair is correlated to a change in risk of disease for the individual. For example, in certain embodiments, an increase in DNA damage or a decrease in DNA repair are correlated to an increased risk of disease for the individual.

In an embodiment, the relative level of DNA damage for the individual as compared to the plurality of control samples is compared to the relative level of DNA repair for the individual as compared to the plurality of control samples and then used to determine if the individual has an increased risk of disease. In certain embodiments of the methods and systems of the present invention, an increase in DNA damage as compared to DNA repair is indicative of an increased risk for disease. Or, a decrease in DNA damage may be indicative of a decreased risk of disease. For example, in an embodiment, an increase in the relative level of DNA damage coupled with a decrease in the relative level of DNA repair is correlated with an increase risk of at least one disease that is associated with DNA damage. Or, a decrease in the relative level of DNA damage coupled with a increase in the relative level of DNA repair may be correlated with an decreased risk of at least one disease that is associated with DNA damage.

In other embodiments, an increase (or decrease) in DNA damage level test results compared to DNA repair results may be indicative of a change in a lifestyle, environmental or physiological variable of interest. For example, in an embodiment, a positive correlation between an increase in DNA damage and the at least one lifestyle, environmental, or physiological variable provides an indication that the at least one variable may be associated with damage to the individual's DNA. Or, a correlation between an increase in DNA repair and the at least one variable may provide an indication that the at least one variable may be associated with damage to the individual's DNA. For example, DNA repair enzymes may be increased by DNA damage. Alternatively, high levels of DNA repair enzymes may indicate that in spite of DNA damage, the DNA is of good quality due to high levels of DNA repair. In an embodiment, an increase in the level of DNA damage that is not coupled with a comparable increase in the level of DNA repair, or a decrease in the level of DNA repair that is not coupled with a comparable decrease in the level of DNA damage, may be indicative of a negative impact of the at least one variable on overall DNA damage accumulation in the individual.

In certain embodiments of the methods or systems of the present invention, a panel of assays is used to measure DNA damage and DNA repair activity in an individual. For example, in an embodiment, measurement of both DNA repair and DNA damage is performed by a plurality of assays for both DNA repair and DNA damage. Or, a plurality of assays that measure specific indicators of DNA damage may be used. Additionally or alternatively, a plurality of assays that measure specific indicators of DNA repair may be used. The measured levels of DNA damage and DNA repair may be compared to reference ranges (e.g., standard curves) for each assay methodology. As described herein, the reference ranges may be derived from the individual being monitored (e.g., before exposure to a particular variable of interest), normal healthy individuals (e.g., age-matched healthy controls), individuals who have experienced a selected environmental, physiological, or lifestyle variable change, or individuals with different diseases.

Assays for measuring DNA damage activity may be those known in the art. Such assays may include quantification of certain DNA adducts. In certain embodiments, DNA adducts may be measured by high performance liquid chromatography with electrochemical detection (HPLC-ECD), or 2-dimensional liquid chromatography with tandem mass spectrometry (2D-LC-MS/MS). Or, assays may be used for quantification of DNA breaks. Examples of DNA break assays include Single Cell Gel Electrophoresis (SCGE, also known as the Comet Assay), the Aldehyde Reactive probe (ARP) Assay, and DNA Ladder Assays. Or, assays that measure the susceptibility of DNA to breakage may be used. In yet other embodiments, measurement of DNA damage may be quantified by determining the levels of expression of selected DNA repair enzymes (DRE).

In various embodiments, the DNA adduct assay comprises measurement of at least one of (but not limited to) 8-hydroxydeoxyguanosine (8OHdG), 8-hydroxyguanosine (8OHG), 8-oxoguanine (8-oxo-G), 2,6-Diamino-4-hydroxy-5-formamidopyrimidine (FapyGua), 8-hydroxy-adenine/8-oxoadenine (8OHAde), O6-methyl-guanine, 4,6-Diamino-5-formamidopyrimidine (FapyAde), 5-hydroxy-cytosine (5-OH-Cyt), 5-Hydroxy-methylhydantoin (5-OH-5-MeHyd), 5-hydroxy-hydantoin (5-OH-Hyd), 2-oxoadenine (2-OH-Ade), and 5-Hydroxy-methyl-uracil (5-OH-Me-Ura).

In some embodiments, at least one of the assays may comprise a measurement of DNA repair. In certain embodiments, the measurement of DNA repair may be quantified by at least one of a DNA Repair Enzyme assay, a Repair Capacity Analysis assay, or a DNA Damage Susceptibility assay. For example, the DNA Repair Enzyme assay may measure expression of at least one of (but not limited to) 8-oxoguanine DNA glycosylase (OGG 1), MutY homolog (hMYH), MutT Homolog-1 (MTH1), Heme oxygenase 1 (HOX1), NEIL endonuclease VIII-like 1 protein (NEIL1 protein), Nth homolog 1 (NTH1 protein), excision repair cross-complementing protein (ERCC1), AP endonuclease (Ape-1), or superoxide dismutase (SOD-1). In an embodiment, enzyme activity may be measured. Or, the levels of enzyme protein may be measured, e.g., using an antibody to the enzyme and an colorimetric immunodetection assay such as an ELISA. In yet other embodiments, the level of DRE gene expression is measured by measuring DRE mRNA levels for specific genes. In an embodiment, the mRNA may be quantified by RT-PCR.

The methods and/or systems of the present invention may be particularly useful for guiding a physician in making decisions regarding treatment protocols and/or in providing feedback to individuals regarding the effects of positive or negative changes in lifestyle variables. In some embodiments, the methods and/or systems of the present invention may comprise determining whether at least one of an environmental, physiological or lifestyle variable has changed for the individual and correlating the results of the measurement of DNA damage and/or repair to that change. For example, the ability of an individual to observe beneficial changes in DNA damage accumulation in response to changes in diet can serve as positive reinforcement for continued compliance with a diet regimen.

Or, the methods or systems of the present invention may be useful for guiding a physician in making decisions regarding the need for additional diagnostic tests and/or monitoring to identify the presence of disease in the individual, and may be used in conjunction with the individual's prior medical history, family history, presenting symptoms, standard diagnostic tests, and the like. Diseases for which an increased risk may be identified in an individual include, but are not limited to, cancers, degenerative diseases, or any disease associated with oxidative stress or DNA degradation. For example, cancers that may be associated with DNA damage, and thus, that may be identified as possible health risks by the methods and systems of the present invention include leukemia; hepatocellular carcinoma; adenocarcinoma; and colorectal, gynocological (including cervical and ovarian), renal and gastric cancers; and cancers of the brain, lung (including squamous cell carcinoma and small cell carcinoma), stomach and colon. Other diseases that may be associated with DNA damage, and thus, that may be identified as possible health risks by the methods and systems of the present invention include: cystic fibrosis; Type II diabetes; hematological disorders; Parkinson's disease; Dementia, including Alzheimers Disease; multiple sclerosis; amyotrophic lateral sclerosis (ALS); cardiovascular disease; chronic hepatitis; HCV liver cirrhosis; H. pylori infection; systemic lupus erythematosus; rheumatoid arthritis; Fanconi's anemia; and conditions such as: Down Syndrome and kidney transplant (Cooke et al. (2003) FASEB J. 17:1195-1214). The ability of a physician to observe beneficial changes in DNA damage accumulation in a patient would provide guidance to the physician and assist with decisions regarding ongoing treatment.

The determination of whether DNA damage or DNA repair level is greater than or less than average levels for controls (e.g., age-matched healthy controls or other selected controls) may involve the determination of a difference of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, or at least 50%, or more between a measurement obtained for an individual using a given DNA damage or DNA repair assay and the control level or reference range considered for comparison for the selected assay. In other embodiments, the differences between the individual being measured and the control level or reference range may be from about 5% to 500%, or from about 10% to about 400%, or from about 15% to about 300%, or from about 20% to about 200%, or from about 20% to about 100%.

Alternatively, the determination of whether DNA damage or DNA repair activity is greater than or less than control values or reference range may involve the determination of whether a measurement obtained for an individual using a given DNA damage and/or DNA repair assay falls outside of the standard error for the corresponding control (e.g., reference range or standard curve) for the selected assay.

In some embodiments, a qualitative comparison of the levels of both DNA damage and DNA repair may be made to obtain an apparent overall measurement of DNA damage accumulation. An increase in the level of DNA damage coupled with a comparable increase in the level of DNA repair may indicate a good state of health, with no apparent increased risk of disease or may alternatively indicate no impact of a selected environmental, physiological, or lifestyle variable on overall DNA damage accumulation. A decrease in the level of DNA damage coupled with a comparable decrease in the level of DNA repair may indicate the same. However, an increase in the level of DNA damage that is not coupled with a comparable increase in the level of DNA repair, or a decrease in the level of DNA repair that is not coupled with a comparable decrease in the level of DNA damage, might indicate health risk or a negative impact of a selected environmental, physiological, or lifestyle variable on overall DNA damage accumulation.

In other embodiments, the methods or systems for predicting increased risk for disease do not involve comparison of measurements to control values or reference ranges for others (e.g., healthy age-matched controls) but may involve comparison to baseline measurements in the individual that is being monitored. For example, in certain embodiments, the methods or systems may comprise: (a) establishing baseline measurements of at least one of DNA damage and DNA repair activities in an individual using a plurality of selected assays of either DNA damage and/or DNA repair; (b) repeating the measurements using the selected assays at one or more later time points; (c) comparing the levels of at least one of DNA damage or DNA repair at each time point to the values obtained as a baseline; and (d) and determining whether the levels of DNA damage and/or repair at later time points has increased or decreased compared to baseline measurements. In certain embodiments of the methods and systems that employ base line analysis, both DNA damage and DNA repair are measured. Thus, in some embodiments, the method may comprise a plurality of assays of DNA damage and/or a plurality of assays of DNA repair. Thus, the method may comprise performing a plurality of assays of DNA damage, and/or a plurality of assays of DNA repair, and/or at least one assay each of DNA damage and DNA repair.

For example, in some embodiments, the methods or systems for predicting increased risk for disease does not involve comparison of measurements to control values or reference ranges for others (e.g., healthy age-matched controls) but may involve comparison to baseline measurements in the individual that is being monitored. For example, in certain embodiments, the methods or systems may comprise: (a) establishing baseline measurements of at least two measurements of DNA damage or two of DNA repair activities in an individual, or one of both using at least one selected assay; (b) repeating the measurements at one or more later time points; (c) comparing the levels of both DNA damage and DNA repair at each time point to the values obtained as a baseline; and (d) and determining whether the levels of DNA damage and/or repair at later time points has increased or decreased compared to baseline measurements. In certain embodiments of the methods and systems that employ baseline analysis, both DNA damage and DNA repair are measured using a plurality of assays for each DNA damage and DNA repair.

The determination of whether DNA damage and repair has increased or decreased according to each test method compared to baseline measurements may involve the determination of a difference of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, or more between the baseline measurement and the measurement at a later time point. In certain embodiments, an increase in the level of DNA damage coupled with a decrease in the level of DNA repair is indicative of an increased risk for disease. In other embodiments, the changes may range from about 5% to 500%, or from about 10% to about 400%, or from about 20% to about 300%, or from about 30% to about 200%, or from about 40% to about 100%.

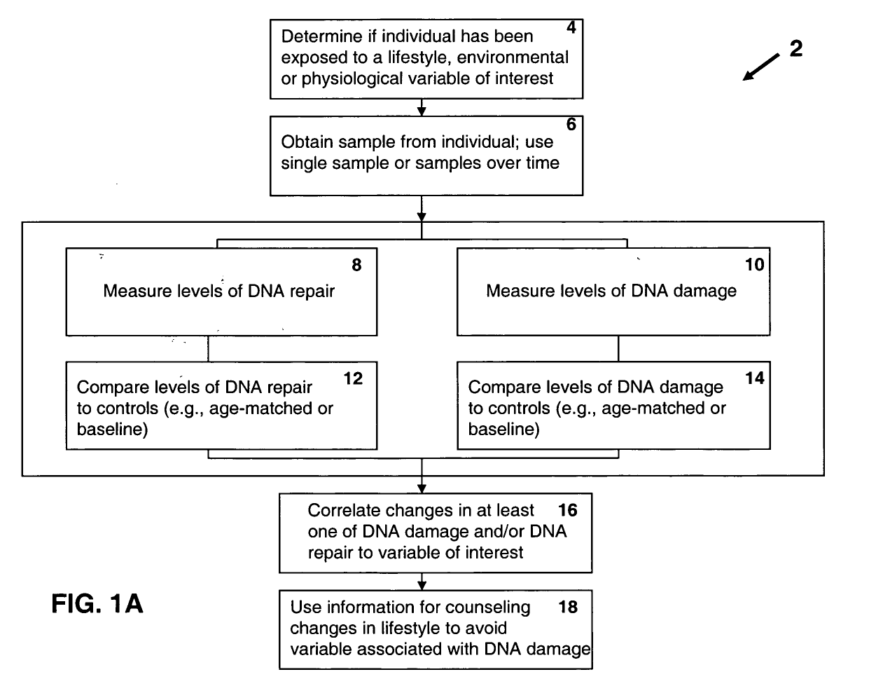

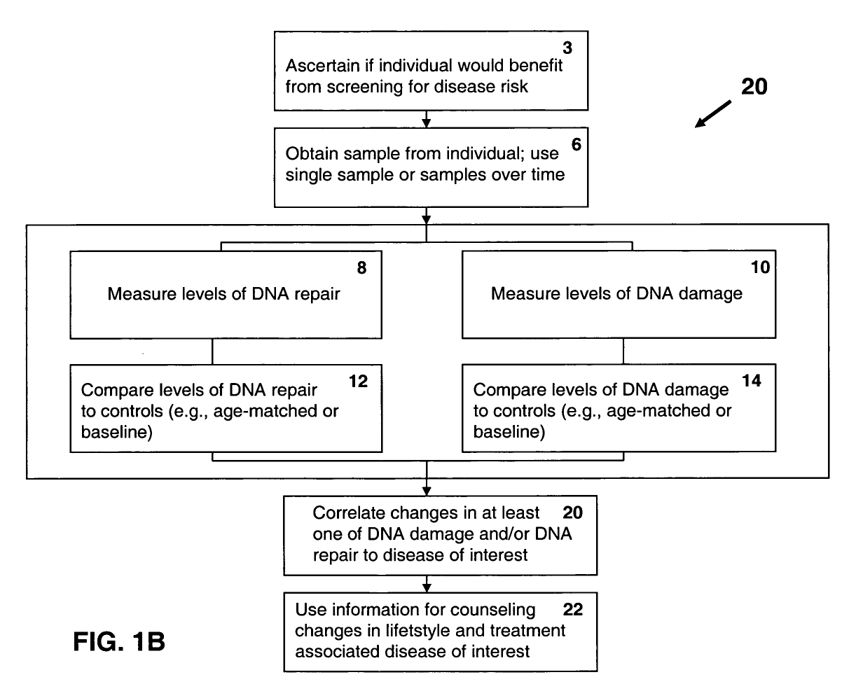

FIG. 1 shows example embodiments of methods 2, 20 of the present invention. The method 2 may be used to determine if DNA damage and/or DNA repair is associated with a variable (e.g., lifestyle, environmental, physiological) of interest (Panel 1A). Or, the method 20 may be used to determine if DNA damage and/or DNA repair is associated with a risk of disease. Thus, in an embodiment, the method 2 (Panel 1A) may comprise the step 4 of interviewing the individual to determine whether the individual has been exposed to certain lifestyle, environmental or physiological variables. Or in an alternate embodiment, the method 20 (Panel 1B) may comprise the step 3 of evaluating the individual as one who would benefit from a determination of disease risk. The methods 2, 20 may further include the step 6 of obtaining a sample from the individual. In some cases a single sample may be obtained. Alternatively, the method may comprise obtaining multiple samples over time from the individual.

The methods 2, 20 may further comprise the steps of measuring DNA repair 8 and/or measuring DNA damage 10. Methods for measuring DNA repair and/or DNA damage may comprise the assays and reagents described herein. Or, other assays and reagents known in the art may be used. The methods 2, 20 may further comprise the steps 12, 14 of comparing levels of DNA repair or DNA damage for the individual to levels of DNA repair or DNA damage for controls. As discussed herein, the controls may comprise a range of values (i.e., reference ranges) derived from a plurality of test subjects. In an embodiment, the reference range may comprise a standard curve. For example, the reference ranges may be derived from healthy, age-matched individuals. Alternatively, the controls may comprise may be a baseline (e.g., time 0) sample (or standard curve) for the individual being monitored. Or, other controls used in the measurement of DNA damage and/or DNA repair may be used.

The method 2 may then comprise the step 16 of correlating the levels of DNA damage and/or DNA repair in the individual to a variable of interest. Or, the method 20 may then comprise the step 20 of correlating the levels of DNA damage and/or DNA repair in the individual to a risk of disease. The results of the testing for DNA damage and/or DNA repair, and the correlation of the results with a variable of interest may then be used for counseling 18 regarding changes in lifestyle or environment to avoid a variable associated with increased DNA damage and/or reduced DNA repair (Panel 1A). Or, the results of the testing for DNA damage and/or DNA repair, and the correlation of the results with a disease risk may then be used for counseling 22 regarding changes in lifestyle and/or medical treatment to reduce the risk of the disease that is associated with increased DNA damage and/or reduced DNA repair (Panel 1B).

In other embodiments, the present invention may comprise systems for measuring DNA damage and/or DNA repair in an individual, and correlating the results to a variable of interest. In an embodiment, the system may comprise reagents for measuring levels of DNA damage and/or DNA repair. The system may further comprise reagents and/or containers for obtaining and storing samples from an individual to be tested. Also, the system may comprise instructions and/or analysis systems for correlating the changes in at least one of DNA repair and/or DNA damage to a lifestyle, environmental or physiological variable of interest, disease risk, or other health risk variables.

In some embodiments, the system may comprise a plurality of physical locations or stations where the sampling, assaying DNA repair and/or DNA damage, compilation of results, and analysis and interpretation of the results are performed. Thus, in certain embodiments, the system may comprise a station for patient sampling and/or storage of the samples. The system may further comprise a station for preparing the samples to be assayed for levels of DNA damage and/or DNA repair. The system may, in certain embodiments, comprise a station or stations for assaying samples for DNA damage, DNA repair, and/or DNA repair enzyme activity. In some embodiments, the system may further comprise a station for the collection of data and/or a station for the analysis of the results. Once the data has been collected, it may be communicated to the individual being tested. Thus, the system may comprise a station for communication of the results to the individual and/or patient counseling.

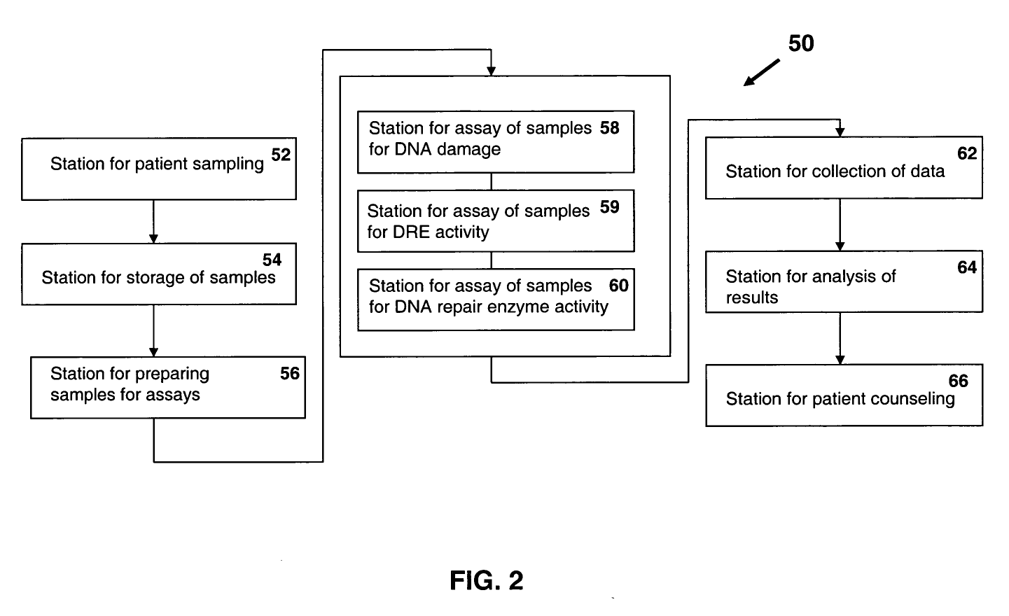

FIG. 2 shows an example of a system 50 of the present invention. For example, in some embodiments, the system may comprise a station 52 for patient sampling. The station for patient sampling may comprise a doctor's office, a clinic or an out-patient site. The system may also comprise a station 54 for storing the patient samples and/or delivering the samples to a location where assays of DNA damage and/or DNA repair may be performed. The system may also comprise a station 56 for preparing the samples for the assay. For example, the samples may need to be enriched by centrifugation, or processed for isolation of protein, DNA and/or RNA.

The system may further comprise stations for the assay of DNA damage 58, DNA repair enzymes 59, and/or DNA repair 60. Thus, as shown in FIG. 2, the system may comprise the assay of DNA damage, and/or the assay of DRE activity, and/or the assay of DNA repair. In various embodiments, the assays of DNA damage, DRE activity and/or DNA repair may be single assays, or a plurality of different assays (i.e., a panel of assays) may be used. The assays may comprise some of the assays as described herein. Or, other types of assays known in the art may be used.

The system may also comprise a station 62 for the compilation of data from the assays of DNA damage and/or DNA repair and/or a station 64 for the analysis of the assay results. The analysis may be provided in a paper format, or may be provided by computer. For example, the system may comprise a computer-based software that allows access to a database of standard curves, reference ranges (for the assay of interest), and/or analysis systems. Alternatively, the system may comprise an access code that may provide a user access to a database of standard curves, reference ranges and/or analysis systems (e.g., over the internet or by a wireless means).

Once the assays have been completed, there may be a need for a health care professional or other individual to provide counseling regarding interpretation of the test results. Thus, the system may comprise a station 66 for patient counseling regarding the test results and providing suggestions for lifestyle changes and/or medical treatments that may be required.

In other embodiments, the system may comprise a kit. The kit may comprise one or a collection of reagents packaged for use by a person or a laboratory wishing to assay DNA repair and/or DNA damage in an individual or individuals. Thus, in alternate embodiments, a kit may comprise a single reagent packaged in a form to be used to measure at least one of DNA damage and/or DNA repair, or a plurality of such reagents.

In certain embodiments, the kit may comprise one or some of the reagents required to perform at least assay of DNA damage or DNA repair. For example, in certain embodiments, the kit may comprise reagents for performing an assay to measure DNA damage. Such reagents may include, for example, compounds used to measure DNA adducts. In certain embodiments, the DNA adducts that may be measured may include 8-hydroxydeoxyguanosine (8OHdG), 8-hydroxyguanosine (8OHG), 8-oxoguanine (8-oxo-G), 2,6-Diamino-4-hydroxy-5-formamidopyrimidine (FapyGua), 8-hydroxy-adenine/8-oxoadenine (8OHAde), 06-methyl-guanine, 4,6-Diamino-5-formamidopyrimidine (FapyAde), 5-hydroxy-cytosine (5-OH-Cyt), 5-Hydroxy-methylhydantoin (5-OH-5-MeHyd), 5-hydroxy-hydantoin (5-OH-Hyd), 2-oxoadenine (2-OH-Ade), and 5-Hydroxy-methyl-uracil (5-OH-Me-Ura).

Or, the reagents in the kit may include compounds to measure DNA breakage. For example, reagents to measure DNA breakage may include compounds to measure DNA mobility by the Comet assay, or to size DNA using a DNA ladder assay, or to measure the number of abasic (i.e., apurinic or apyrimidinic (AP)) sites in DNA (e.g., using an ARP assay).

For example, in certain embodiments, the reagents may comprise primers to quantify DNA breakage by a DNA ladder assay. In certain embodiments, the primers and/or probes may comprise at least one oligonucleotide from sequences publicly available such as primers having the sequence as set forth in SEQ ID NOs: 1-14. Or sequences at least 70%, 75%, 80%, 85%, 90%, 95%, 96%, 97%, 98%, 99%/o identical to the sequences as set forth in SEQ ID NOs: 1-14 may be used.

In other embodiments, the kit may comprise reagents for performing an assay to measure DNA repair. Such reagents may include, for example, primers and/or probes to measure the levels of DNA repair enzyme (DRE) mRNA. In another embodiment, enzyme activity may be measured. Or, the levels of enzyme protein may be measured, e.g., using an antibody to the enzyme and a colorimetric immunodetection assay such as an ELISA. In various embodiments, the repair enzymes measured by the assays may comprise 8-oxoguanine DNA glycosylase (OGG1), MutY homolog (hMYH), MutT Homolog-1 (MTH1), Heme oxygenase 1 (HOX1), NEIL endonuclease VIII-like 1 protein (NEIL1 protein), Nth homolog 1 (NTH1 protein), excision repair cross-complementing protein (ERCC 1), AP endonuclease (Ape-1), or superoxide dismutase (SOD-1).

For example, in certain embodiments, the reagents may comprise primers and/or probes for detection of DRE mRNA. In certain embodiments, the primers and/or probes may comprise at least one oligonucleotide having the sequence as set forth in SEQ ID NOs: 15-44. Or sequences at least 70%, 75%, 80%, 85%, 90%, 95%, 96%, 97%, 98%, 99% identical to the sequences as set forth in SEQ ID NOs: 15-44 may be used. For probes, oligonucleotide may include fluorophores or quenching moieties as shown for SEQ ID NOs: 17, 20, 23, 26, 29, 32, 35, 38, 41, and 44 as described herein. Or, probes without such moieties, or with different detection agents may be used. Alternatively or additionally, the reagents may include reagents to perform the DNA Repair Capacity Assay (RCA) or the DNA Damage Susceptibility Assay.

In some embodiments, the kit may also contain containers and/or reagents for collecting samples. For example, the kit may contain tubes that allow for rapid procurement of urine, blood, saliva or other bodily samples. The sampling containers/devices may comprise the addition of preservatives or other chemicals that are known to preserve the integrity of DNA (e.g., EDTA) or RNA (e.g., RNAsin).

The kit may also contain instructions for interpreting the results of the assays. For example in an embodiment, the kit may comprise reference ranges that may be used to evaluate the results obtained for the individual being tested. Or, the kit may provide instructions for correlating the measured results to a change in a variable of interest or a disease risk. The instructions may be provided in a paper format, or may be accessed by computer. For example, the kit may comprise a CD-ROM or other media having a computer-based software that allows access to a database of standard curves and/or analysis systems. Alternatively, the kit may comprise an access code that may provide access to a database of standard curves and/or analysis systems (e.g., over the internet or by a wireless means).

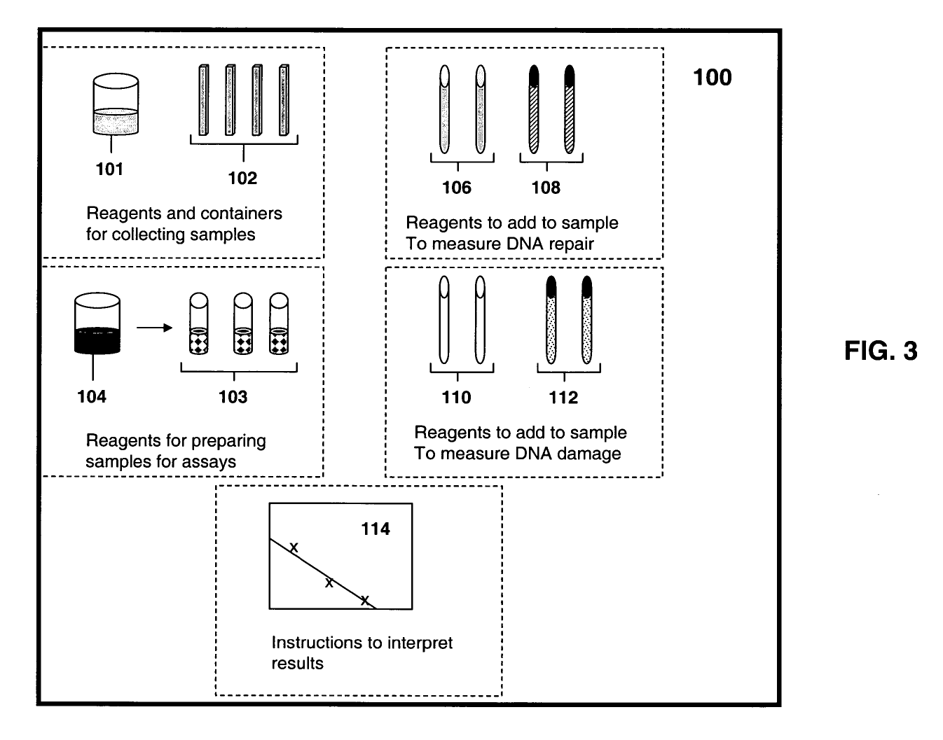

An embodiment of a kit 100 of the present invention is shown in FIG. 3. Thus, in an embodiment the kit may comprise reagents 101 and/or containers 102 for collecting samples. Also, the kit may comprise reagents 104 that may be added to the collected samples 103 to prepare the samples for an assay of interest (e.g., to precipitate RNA or DNA). Also, the kit may include reagents 106, 108 to add to the sample to measure DNA repair. Such reagents may provide a result that can be monitored visually (e.g., by ELISA), or in a form for quantifying using a measurement device (e.g., fluorescence measurement of real-time PCR of DNA repair enzyme levels). The kit may also comprise reagents 110, 112 to measure DNA damage. Such reagents may provide a result that can be monitored visually, or in the form to be quantified using a measurement device (e.g., 2D-LC-MS/MS or antibody measurement of 8-hydroxydeoxyguanosine in urine). The kit may also comprise instructions to perform the assays and/or instructions 114 for interpretation of the results. For example, the kit may comprise reference ranges that may be used to evaluate the results obtained for the individual being tested. Or, the kit may provide instructions for correlating the measured results to a change in a variable of interest or a disease risk. The instructions may be on paper. Or, as described herein, the kit may comprise computer software to provide interpretation analysis or to allow the user to access software and/or databases required for interpretation of the results.

Assays Used to Measure DNA Damage or DNA Repair

In various embodiments, the methods and systems of the present invention use a variety of assays to measure both DNA damage and DNA repair activity. The assays may be used singly or in combination. Bodily fluids and tissues in which DNA, mRNA, or protein is relatively stable may be used for measurement of DNA damage and/or DNA repair. Such tissues may include whole blood, serum, plasma, leukocytes, urine, buccal swab samples, whole tissue samples, and cerebrospinal fluid.

i. DNA Damage

In certain embodiments, the methods and systems of the present invention may comprise measurement of DNA damage. The DNA damage assays may comprise the assays described herein. Or, other DNA damage assays may be employed. In certain embodiments, assays for measuring DNA damage may comprise high performance liquid chromatography (HPLC). In an embodiment the HPLC may be used with electrochemical detection (ECD). In other embodiments, the assay for DNA damage may comprise two-dimensional liquid chromatography with tandem mass spectrometry (2D-LC-MS/MS) to identify and quantify DNA adducts. Other assays that may be used to monitor DNA damage include the quantification of DNA breaks by a Comet Assay, an ARP Assay, or DNA Ladder Assays.

In yet other embodiments, DNA damage may be measured by quantifying the expression of at least one DNA repair enzyme (DRE). For example, the levels of expression of such DREs may be measured by RT-PCR. Or, the level of expression of such DREs may be measured by measuring the amount of a specific DRE protein. Or, enzyme activity assays may be used. Alternatively or additionally, DRE quantification can also be considered to be a measurement of DNA repair, as described elsewhere herein.

A. DNA Adduct Assays

For example, in one embodiment, measurement of DNA damage may comprise the measurement of DNA adducts. DNA adduct assays involve the measurement of the level of a given altered DNA base or nucleotide associated with DNA damage in various bodily fluids and tissues of an individual. Methods for the detection of such adducts may include, but are not limited to HPLC, HPLC-ECD, liquid chromatography (LC), mass spectrometry (MS), or combinations of such measurement techniques, e.g., two dimensional liquid chromatography tandem mass spectrometry (2D-LC-MS/MS), and the like.

For example, in certain embodiments of the present invention, the DNA adduct to be measured may be at least one of 8-hydroxydeoxyguanosine (8OHdG), 8-hydroxyguanosine (8OHG or 8OHGua), or 8-oxoguanine (8-oxo-G). Oxygen free-radicals can preferentially oxidize guanine (G) bases in DNA to 8-oxo-G. If this oxidation is not repaired, the oxidation can lead to a mutation due to mispairing of the mutated strand in double-stranded DNA. For example, instead of its normal bond with cytosine (C), 8-oxo-G hydrogen-bonds with thymine (T). This can lead to a change in the DNA sequence when the strand containing the 8-oxo-G is copied, resulting in a mutation from C to T on the complementary strand. When this complementary strand is copied, the original G is now replaced with an A, resulting in an A-T transversion. However, the human DNA repair system involves enzymes that constantly scan active DNA for oxidative damage. In base excision repair (BER), a DNA repair enzyme (glycosylase) detects an 8-oxo-G and it removes the 8-oxo-G from the DNA strand. The excised 8-oxo-G base is excreted in urine. Alternatively, if the damage is repaired by nucleotide excision repair (NER), a contiguous stretch of 5-30 nucleotides is removed. In this case, the excised oligomer is degraded into component nucleotides, which may include 8OHdG, and these are excreted in urine.

Thus, in certain embodiments of the methods and systems of the present invention, measurement of 8-hydroxydeoxyguanosine or 8-oxoguanine in urine and other bodily fluids and/or tissues therefore provides a means for monitoring oxidative DNA damage by measuring repair products. Assays for measuring urinary 8-hydroxydeoxyguanosine are known in the art. Such assays may employ, for example, a series of solid-phase extraction steps that separate 8-hydroxydeoxyguanosine from other urinary constituents, followed by analysis by gradient reversed-phase HPLC coupled to a dual-electrode high-efficiency ECD system (Shigenaga et al. (1989) Proc. Natl. Acad. Sci. USA 86:9697-9701). Or, two-dimensional liquid chromatography tandem mass spectrometry (2D-LC-MS/MS), may be used to identify and quantify the adducts (e.g., Ravanat et al. (1998) J. Chromatography B 715:349-356; Weimann et al. (2001) Free Radical Biology & Medicine 30:757-764) (also see e.g., Example 1).

The levels of any DNA adduct that may be associated with DNA damage may be measured as part of the methods, systems or kits of the present invention. Thus, in addition to the guanosine adducts described above, other adducts measured using the methods, systems and kits of the present invention may include 2,6-diamino-4-hydroxy-5-formamidopyrimidine (FapyGua), 8-hydroxy-adenine/8-oxoadenine (8OHAde), 06-methyl-guanine, 4,6-diamino-5-formamidopyrimidine (FapyAde), 5-hydroxy-cytosine (5-OH-Cyt), 5-hydroxy-methylhydantoin (5-OH-5-MeHyd), 5-hydroxy-hydantoin (5-OH-Hyd), 2-oxoadenine (2-OH-Ade), and 5-hydroxy-methyl-uracil (5-OH-Me-Ura) (Cooke et al. (2003) The FASEB Journal, 17:1195-1214; Loft & Poulsen (1999) Methods in Enzymology, 300:166-184).

B. DNA Breakage

In other embodiments of the methods, systems and kits of the present invention, DNA damage may be measured by assessing DNA fragmentation by the quantification of DNA breaks. Assays that may be used to quantify DNA breaks may include the Comet Assay, the DNA Ladder Assay, and/or the ARP Assay.

For example, in one embodiment, the Comet assay is used to measure DNA breaks in a sample. In certain embodiments, the Comet Assay allows actual visualization of the DNA in individual cells. In certain embodiments, the Comet assay uses electrophoresis of single cells to monitor DNA breakage levels. During single-cell gel electrophoresis, DNA molecule fragments (e.g., smaller molecules compared to intact chromosomal DNA) can migrate from within the cell. Intact DNA is unable to migrate very far, while damaged DNA can exit the cell and form a “comet” pattern (see, e.g., Collins (2004) Mol. Biotechnol., 26:249-261).

In another embodiment, a DNA ladder assay may be used to monitor DNA breakage. In an embodiment, agarose gel electrophoresis may be used to measure fragmented DNA in cells. In agarose gel electrophoresis, fragmented DNA, which is essentially a collection of molecules of various molecular weights, appears as a ladder when the molecules are separated by size on a gel. In an embodiment, the degree of fragmentation correlates with degree of DNA damage.

The degree of DNA damage may also be monitored using a DNA ladder assay in combination with PCR. The PCR-Ladder assay measures the ability of the DNA to function as a substrate for PCR by visualizing the amplification products as DNA ladders on a gel. In the PCR-DNA Ladder Assay, PCR amplification of segments of different lengths of several genes (e.g., β-globin and/or the entire mitochondrial genome) allows for visualization of the PCR product as a DNA ladder on a gel, and quantification of damage. The gene selected for amplification is not particularly crucial, although the assay may provide more information where there are a plurality of loci to be amplified (e.g., amplification of the mitochondrial genome). The same gene (or genome) may be used as a template for different-sized amplicons. Generally, amplification of short amplicons is easier than amplification of longer amplicons and therefore, short amplicons are detected in relatively high concentration. With increasing DNA damage, however, longer amplicons become even more rare, since the PCR template will be broken somewhere along its length and no longer serves as a full-length template. When such breakage occurs, the pattern on the agarose gel will be that of a ladder heavily weighted towards shorter amplicons, with the large amplicons either absent or giving faint bands. In undamaged DNA, more bands of longer lengths are generally visible. By quantifying band image density between undamaged controls and damaged samples, a DNA fragmentation quantitative result may be obtained (e.g., Example 2).

Another DNA damage assay that may be used is the aldehyde reactive probe (ARP) assay. The ARP assay is a simple, rapid, and sensitive method for the detection of abasic (i.e., apurinic or apyrimidic) (AP) sites caused by base removal in damaged DNA. The biotinylated aldehyde-specific reagent, ARP, reacts specifically with the aldehyde group present in AP sites, resulting in biotin-tagged AP sites in DNA. The biotin-tagged AP sites can then be quantified calorimetrically against standards with known numbers of AP sites with an ELISA-like assay, using avidinfbiotin-conjugated horseradish peroxidase as the indicator enzyme (see, e.g., Kow & Dare (2000) Methods, 22:164-169 and BioVision Research Products' DNA Damage Quantification Kit).

ii. DNA Repair

A. Measurement of DNA Repair Enzymes (DRE)

In some embodiments, the assays may comprise measurement of the level of DNA repair enzymes (DREs) and repair-related enzymes. Increases levels of DREs and repair-related enzymes can be an indication of increased DNA damage, which leads to the up-regulation of expression of such enzymes. Or, increases in DREs and repair-related enzymes may be indicative of increased repair, and improved DNA quality. There are a variety of different DNA adducts that may be formed in the cell and require repair by specific DNA repair enzymes. DNA repair may be categorized by the mechanism of repair. For example, there are two well-defined forms of DNA damage repair: Base Excision Repair (BER) and Nucleotide Excision Repair (NER).

The pathway for BER generally involves several steps. First, the altered base on the DNA is recognized and removed by a specific glycosylase DRE, which removes the base from the sugar backbone, leaving an apurinic or apyrimidic site (AP site). Second, an AP endonuclease recognizes the AP site and hydrolyzes the 5′ phosphodiester bond that joins the baseless sugar to the rest of the DNA molecule. Next, the remaining deoxyribosephosphate residue is removed by a phosphodiesterase, which cleaves the 3′ phosphodiester bond, releasing the sugar and leaving a small gap in the DNA helix. DNA polymerase may then bind to the 3′ end of the cut DNA strand and fill in the gap by making a complementary copy of the information stored in the template strand. Finally, the break in the damaged strand left when the DNA polymerase has filled in the gap is sealed by DNA ligase.

In contrast to BER which recognizes a single altered base, NER works through the activity of enzymes that recognize distortions in the DNA double helix, such as distortions caused by large bulky adducts (e.g., binding the carcinogen benzopyrene), or other types of chemical changes in the DNA structure (e.g. pyrimdine dimers induced by sunlight). The enzymes are not specific in detecting individual damaged bases, but instead excise contiguous stretches of nucleotides in the area of the damage by cleaving the abnormal strand on each side of the distortion. Such excised oligomers may be 5-30 nucleotides in length. A DNA helicase enzyme peels the oligomer off its partner strand and the large gap left is repaired by DNA polymerase and DNA ligase, as for a single nucleotide gap in BER.

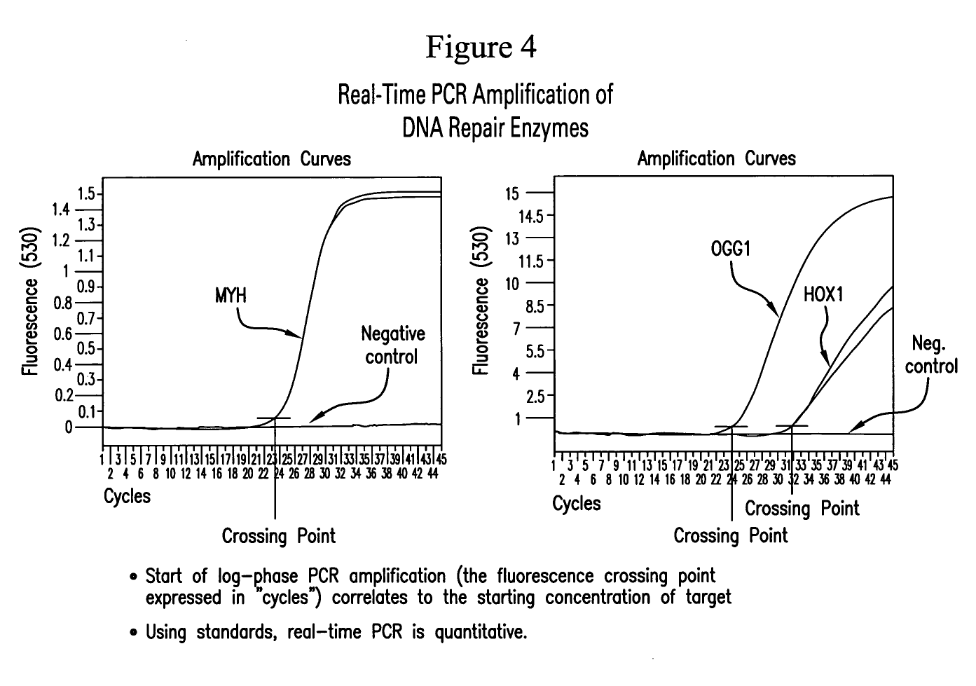

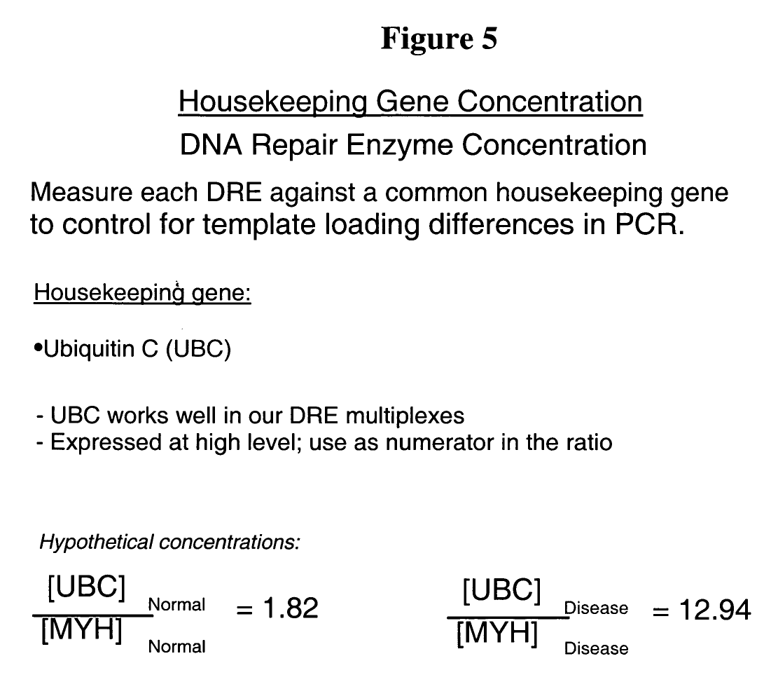

As DNA damage occurs, DNA repair and repair-related enzymes may be up-regulated over their basal level of expression. Such up-regulation can be detected using assays to measure DREs and repair-related enzymes. For example, expression of genes encoding such DREs or repair-related enzymes may be quantified by measuring mRNA levels for the DRE or repair-related enzyme of interest using RT-PCR (FIG. 4; Example 3) and comparing the measured levels of the DRE or repair-related enzyme mRNA to the mRNA levels of a housekeeping gene (FIG. 5). Changes in the ratio of the DRE or repair-related enzyme mRNA as compared to the mRNA for the housekeeping gene may indicate that the individual is at increased risk for DNA damage. Conversely, changes in the ratio of the DRE or repair-related enzyme mRNA as compared to the mRNA for the housekeeping gene may indicate a decreased risk, especially if coupled with results from another test indicating decreased damage coupled with increased DRE or repair-related enzyme expression. The levels of the DRE as measured in the individual can be compared to known control samples using age-matched reference ranges or (e.g., a standard curve correlating age with DRE levels). The levels of the DRE or repair-related enzyme as measured in the individual can be quantified by comparing to known control samples using a standard curve.

A number of DNA repair and repair-related enzymes have been identified and may be measured using DRE assays within the methods of the present invention. Such DNA repair and repair-related enzymes include, but are not limited to:
1. OGG1—The specific glycosylase OGG1 removes 8-oxo-G from DNA (Fortini et al. (2003) Mutat. Res. 531:127-139; Boiteux & Radicella (1999) Biochimie 81:59-67);
2. MutY homolog (hMYH)— hMYH is a repair enzyme for 2-hydroxyadenine in DNA (Ohtsubo et al. (2000) Nucleic Acids Res. 28:1355-1364);
3. MutT Homolog-1 (MTH1)— MTH1 hydrolyzes oxidized purine nucleoside triphosphates, such as 8-oxo-dGTP, 8-oxo-dATP, and 2-hydroxy (OH)-dATP (Nakabeppu (2001) Prog. Nucleic Acid Res. Mol. Biol. 68:75-94);
4. Heme oxygenase-1 (HOX 1)—HOX 1 is a 32 kDa stress protein that degrades heme to biliverdin, free iron and carbon monoxide (Schipper (2000) Exp. Gerontol. 35:821-830);
5. Endonuclease VIII (NEIL 1 protein)—NEIL 1 protein excises many oxidized pyrimidines (Laval (1996) Pathol. Biol. (Paris) 44:14-24);
6. Nth homolog 1 (NTH protein)—as with NEIL 1 protein, NTH protein excises many oxidized pyrimidines (Marenstein et al. (2003) J. Biol. Chem. 278:9005-9012);
7. Excision repair cross-complementing protein (ERCC1)—ERCC1 has a leading role in nucleotide excision repair because of its damage recognition and excision ability (Altaha et al. (2004) Int. J. Mol. Med. 14:959-970);
8. AP endonuclease (Ape-1)—Ape-1 cleaves deoxyribose-5′ phosphate left behind after a damaged base has been excised by specific glycosylases such as OGG1, MYH1, NTH1 and NEIL1 (Ramana et al. (1998) Proc. Natl. Acad. Sci. USA, 95:5061-5066); and
9. Superoxide dismutase (SOD-1)—SOD-1 prevents oxidative damage to DNA rather than repairing it, by reducing free superoxide radicals, generating hydrogen peroxide which can then be further reduced to water (Fridovich (1978) Science, 201:875-880; Petkau et al. (1975) Biochem. Biophys. Res. Comm., 67:1167-1174). SOD-1 is therefore a repair-related enzyme. Quantitation of SOD-1 levels is currently offered as a stand-alone marker of oxidative challenge in people by BioHealth Diagnostics (San Diego, Calif.).

Although DRE assays may be used as described above to measure DNA damage, such assays may also be used to quantify DNA repair levels. Although the methodology is the same, in the context of measuring DNA repair, up-regulation of repair and repair-related enzyme expression may be indicative of increased DNA repair activity (i.e., more enzyme signals an increase in the amount or level of active repair taking place).

B. Repair Capacity Analysis (RCA)

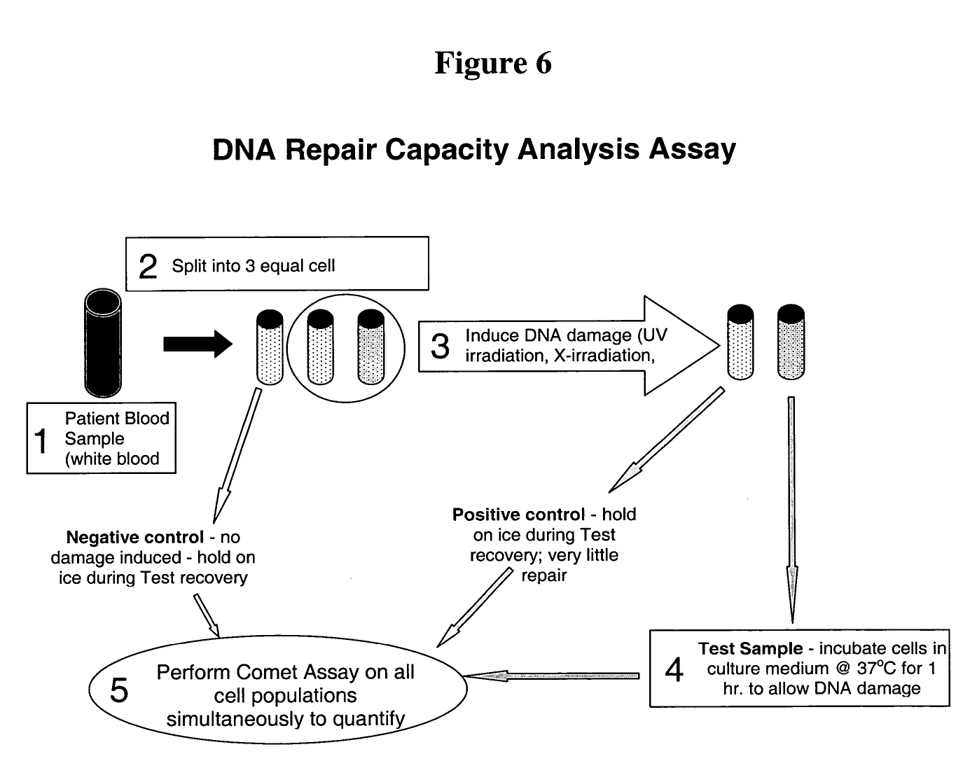

Alternatively or additionally, the Repair Capacity Analysis (RCA) may be used to quantify DNA repair. The RCA assay is a test that may be used to measure cellular repair of DNA damage induced in vitro (see FIG. 6). In an embodiment of the assay, patient cell samples (e.g., leukocytes or cells from solid tissues) may be split into negative controls, positive controls and test samples. The test sample and positive control may be challenged in vitro using a DNA-damaging agent. In alternate embodiments, the DNA damaging agent may comprise at least one of H₂O₂, methylmethane sulfonate, phleomycin, bleomycin, carboplatin, X-ray or ultraviolet-radiation. Or, other DNA-damaging agents known in the art may be used. The test sample is allowed a recovery period after challenge with the DNA-damaging agent; the positive control is tested immediately after treatment with the DNA-damaging agent; and the negative control is either not treated (e.g., no X-ray) or is only exposed to a vehicle (e.g., water in place of H₂O₂). DNA damage is then analyzed using the Comet Assay. Test sample damage may be compared to levels found in negative and positive controls and the degree of repair is reported as a percentage recovery (with the total difference between negative and positive results being 100%) of the total damage induced in the positive control.

C. DNA Damage Susceptibility Assay

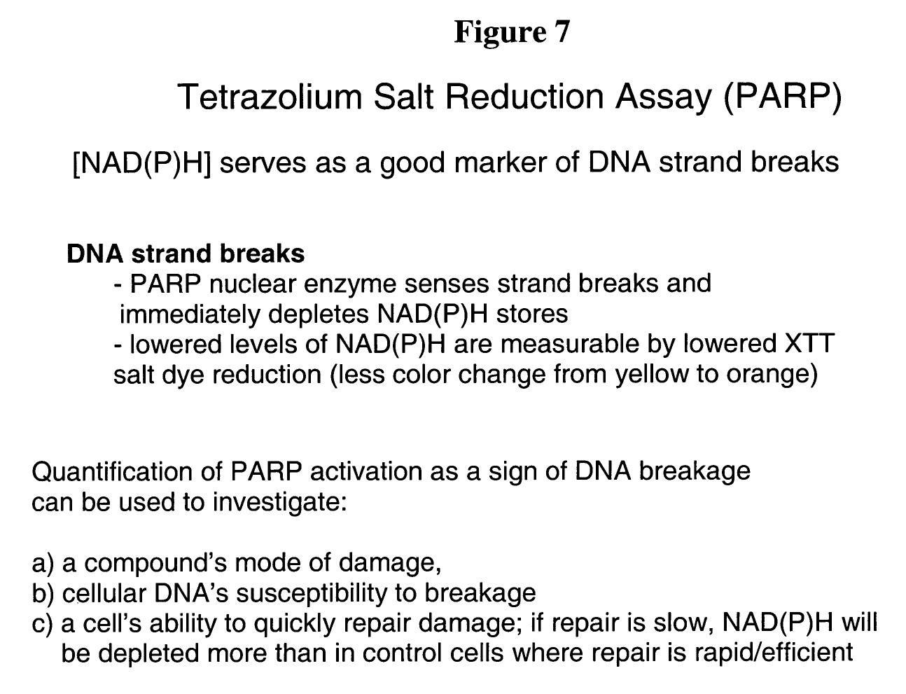

In other embodiments, DNA damage may be measured using a DNA Damage Susceptibility Assay (FIG. 7). In general, the DNA Damage Susceptibility Assay works by measuring the activation of Poly (ADP-ribose) polymerase (PARP), a nuclear enzyme that contributes to DNA repair and is activated by strand breaks in DNA. Activated PARP hydrolyzes NAD(P)H into nicotinamide and ADP-ribose, and polymerizes the ADP-ribose onto nuclear proteins. The level or amount of damaged DNA may be directly proportional to the level of activated PARP and the related and proportional reduction in NAD(P)H levels (see, e.g., Nakamura et al. (2003) Nucl. Acids Res., 31:e104). Therefore, in certain embodiments of the present invention, measurement of NAD(P)H levels can serve as a sensitive assay for the measurement of DNA strand breaks. Low levels of damage susceptibility measured by this test may indicate either: (a) an innate resistance to induction of DNA damage; or (b) rapid repair mechanisms that operate within the timeframe of the assay procedure; or (c) both (a) and (b). Therefore, this assay may be used to monitor DNA repair alone, or as part of a panel of assays as described herein.

### EXAMPLES

The following examples are offered by way of illustration and not by way of limitation.

### Example 1

### Quantitation of 8-Hydroxydeoxyguanosine in Urine

A quantitative, non-invasive measurement of 8-hydroxydeoxyguanosine (8-OH-dG) in urine can be used to monitor oxidative damage to DNA. Quantitative measurement of 8-OH-dG in urine (or other bodily samples) is performed by two-dimensional liquid chromatography with tandem mass spectrometry detection (2D-LC-MS/MS) after sample dilution. Alternatively, 8-OH-dG may be immunoprecipitated from samples of interest and the 8-OH-dG detected by HPLC using methods known in the art. For urine samples, 8-OH-dG concentration is reported as a fraction relative to creatinine level in the sample to normalize for urine concentration (i.e.—8-OH-dG in ng/mg creatinine). Urinary creatinine measurement is a standard and common clinical laboratory test.

For detection by 2D-LC-MS/MS, samples, standards and controls are diluted ten-fold with 1 ng/mL internal standard (stable, isotopically-labeled ¹⁵N₅-8-oxo-2dG) solution in 2% aqueous formic acid for assay. For example, in a typical assay, 100 μL of test sample urine, 100 μL of each of 7 urine standards (e.g., charcoal-stripped pooled urine spiked with 8-OH-dG to 0.1, 0.25, 1.0, 5.0, 10.0, 25.0, 50.0 ng/mL), and 4 Quality Controls (pooled urine samples spiked to ≦0.30, 3.0, 25.0, 40.0 ng/mL 8-OH-dG) are each added to 900 μL of internal standard solution in a multiwell plate. Double-blanks (i.e., no target or internal control) consist of 1000 μL of water. 100 μL of water is added to 900 μL of internal control solution to create blanks (internal control with no target present). The contents of each plate are mixed well, then centrifuged (3700 rpm, 10° C., 10 minutes).

Samples are then run on a Cohesive Technologies/Thermo Aria TX2 or TX4 TurboFlow HTLC System with Aria OS Version 1.5.1 or greater. The system consists of an HTS Twin PAL System Autosampler (CTC Analytics AG) and 2 or 4 (TX2 or TX4, respectively) of each of the following: quaternary pumps, binary pumps, vacuum degassers. Analyte isolation is achieved by gradient separation on first a YMC ODS-3, 50×4.6 mm, 5 μm HPLC column, followed by a Fluophase PFP, 50×4.6 mm, 5 μm HPLC column. Mobile phases consist of: Loading Pump A, 95:5 water:methanol with 0.1% formic acid; Loading Pump B, 5:95 water:methanol with 0.1% formic acid; Eluting Pump A, water; Eluting Pump B, 10:90 water:acetonitrile. Needle wash solutions for the autosampler include: #1, 1% formic acid and #2, 70:30 acetonitrile:1N ammonium hydroxide.

For an automated analysis, the program steps are as follows:
1. The sample is injected onto the first column (YMC ODS-3, 50×4.6 mm) at a flow rate of 0.7 ml/min, and 95% Mobile Loading A;
2. At 20 seconds, a linear gradient of 5% to 25% Mobile Loading B is applied, continuing for 169 seconds, with the flow continuing to waste;
3. At 189 seconds, a linear gradient of 25% to 30% Mobile Loading B is applied, continuing for 45 seconds. The flow from the first column is teed to the Eluting Pump system during this step. After the sample has moved from column 1 to column 2, the Loading Pump is set to wash and re-equilibrate for the remainder of the run time.
4. At this point, the sample has moved from the first column onto the second column (Thermo PFP, 50×4.6 mm)—The Eluting Pump system has a flow rate of 1.0 ml/min and 100% Mobile Eluting A for 20 seconds;
5. At 254 seconds, a linear gradient of 0% to 50% Mobile Eluting B is applied for 90 seconds, with the flow continuing to waste.
6. At 344 seconds, a linear gradient of 50% to 100% Mobile Eluting B is applied for 10 seconds, eluting the sample from the column. The eluent is moved to the mass spectrometer for quantification.
7. At 354 seconds, the flow rate of the Eluting Pump is increased to 1.5 ml/min and 100% Mobile Eluting B for 60 seconds to wash the column, with the flow continuing to waste. Column 2 continues to wash for the duration of the run, which ends at 474 seconds.

An MDS-Sciex API5000 triple quadrupole mass spectrometer operating in positive ion electrospray (ESI) mode with an MDS-Sciex Turbo V™ Ion Source with Turboionspray probe is used for detection. Electrospray ionization is performed in positive ion mode. Nitrogen is used as the nebulizing, curtain, heater, and collision gas. The electrospray probe temperature is set at 450° C. Quantification of analyte and internal standard is performed in selected reaction monitoring mode (SRM). Two transitions are monitored for 8-OH-2dG and two for the internal standard, ¹⁵N₅-8-oxo-2dG (in amu): 284.2→168.1 and 284.2→139.9 for 8-OH-2dG, and 289.2→173.0, and 289.2→145.2 for ¹⁵N₅-8-oxo-2dG. The total mass spec acquisition time is 1 minute. Using Applied Biosystems Analyst software Version 1.5 or greater, the back-calculated amount of 8-oxo-2-deoxyguanosine in each sample is determined from the linear regression curve formed from results obtained for duplicate calibrators (standards) analyzed with each assay.

### Example 2

### DNA Ladder Assay

A PCR-based system is used to determine the relative frequency of damage/breaks in an individual's DNA. By performing short, e.g., ˜400 base pair (bp) to long, e.g., ˜18,000 bp PCR amplifications, the relative number of DNA breaks can be estimated. The procedure relies on the fact that DNA strands with breaks and certain kinds of damage inhibit the completion of a full-length template copy during PCR. Additionally, the longer an amplicon is, the more likely it is that damage will be encountered. Therefore, individuals with an increased amounts of damage will have a reduced amount of the longer expected amplicons produced.

DNA is isolated from cells using the Qiagen Blood & Cell Culture DNA Mini Kit, according to the manufacturer's instructions. DNA is resuspended in 200 μl TE buffer and the quality of the DNA is checked by running 5 μl on an 0.8% agarose gel with molecular weight markers. The concentration of DNA is then determined and approximately equal amounts of DNA from each individual are added in short-amplicon to long-amplicon PCR amplifications containing the following final reagent concentrations: 85 mM potassium acetate, 25 mM tricine pH 8.7, 8% glycerol, 1% dimethylsulfoxide, 1.2 mM magnesium acetate, 0.2 mM each dNTP, 600 nM each primer, 5U Tth DNA polymerase, and 0.02U Vent DNA polymerase. The primers utilized are shown in Table 1 and are specific for the mitocondrial genome and/or β-globin genes with amplicon sizes ranging from 400 bp to 18,000 bp. Or, primers from other genes could be used. PCR conditions are as follows: 1 cycle at 94° C. for 3 minutes; 35 cycles at 72° C. for 1-12 minutes followed by 94° C. for 1 minute; 1 cycle at 72° C. for 1-12 minutes. The specific parameters for each cycle (time/temperature) may be varied as required depending upon the primer sequences, the amount of DNA in the sample, and size of the amplified product.

Amplicons are analyzed by Southern blotting methods or similar techniques for quantification of DNA separated by gel electrophoresis. Estimates of the amount of each amplicon generated are determined performing spot densitometry and comparing sample band intensities to that of a set of standards also included on the run. The relative amounts of DNA damage are determined by comparing the amount of the longer amplicons to the amount of the shortest amplicon. Specifically, the relative amount of damage will be the sum of the ratios of each amplicon, except the shortest, divided by the amount of the shortest amplicon. By comparing the sum obtained in a test sample to a normal, undamaged sample run concurrently (as a normalizing factor), a quantitative result of DNA damage will be obtained.

Older individuals, those with unhealthy lifestyle habits (i.e. smoking, poor diet), or those suffering from some diseases may have more DNA breaks, which will result in fewer long amplicons being formed. The reduction in longer amplicons may be demonstrated by fewer PCR products for the longer amplicons (e.g., weaker bands for larger PCR products when run on a gel). Therefore, the sum of their ratios (normalized against the control run concurrently) will be smaller as compared to individuals with fewer breaks.

TABLE 1

Primers Specific for Mitochondrial and/or
β-Globin Genes

| | | | |
|---|---|---|---|
| | | | Ampli- |
| | | SEQ | con |
| | | ID | Size |
| Primer | Sequence | NO: | (bp) |

| | | | |
|---|---|---|---|
| BGL 1A | GAG ACG CAT GAG ACG TGC A | 1 | |
| BGL 1B | ACA CCT CTA TCC AGC ATC AAC TT | 2 | 426 |
| BGL 1A | GAG ACG CAT GAG ACG TGC A | 1 | |
| BGL 1C | GGT CTT GGG TAC AGG AGT TTG A | 3 | 4387 |
| BGL 4A | TTG TTT GAG ACG CAT GAG ACG TGC | 4 | |
| BGL 4C | AAA TCT TAG AAT GTG TTT GTG AGG | 5 | 8207 |
| | GAG GAA | | |
| BGL 4A | TTG TTT GAG ACG CAT GAG ACG TGC | 4 | |
| BGL 4C | GTT CCC TTG CTT TTC TCT TTT CCC AT | 6 | 12188 |
| BGL 1A | GAG ACG CAT GAG ACG TGC A | 1 | |
| BGL 1F | CAC TGG CTT AGG AGT TGG ACT T | 7 | 17708 |
| MIT 1A | GTC CCA CCC TCA CAC GAT T | 8 | |
| MIT 1B | TTG GCT TAG TGG GCG AAA | 9 | 452 |
| MIT 1A | GTC CCA CCC TCA CAC GAT T | 8 | |
| MIT 1D | GGT AGA TGT GGC GGG TTT TA | 10 | 4818 |
| MIT 4A | GTC CCA CCC TCA CAC GAT TCT TTA CC | 11 | |
| MIT 4B | TAT GGG AGA TTA TTC CGA AGC CTG GT | 12 | 7983 |
| MIT 4A | GTC CCA CCC TCA CAC GAT TCT TTA CC | 11 | |
| MIT 4C | GGT CGG AGG AAA AGG TTG GGG A | 13 | 12248 |
| MIT 1A | GTC CCA CCC TCA CAC GAT T | 8 | |
| MIT 1F | CGT GAA GGT AGC GGA TGA TT | 14 | 16307 |

### Example 3

### DNA Repair Enzyme Assays

Constitutively expressed DNA repair enzymes exist to quickly repair DNA lesions before they can cause permanent mutations. Decreased repair activity is therefore associated with adverse events such as disease development and accelerated aging. Measurement of gene expression levels by RT-PCR of a selected panel of DNA repair enzymes is performed as described below.

Two protocols are provided for extracting mRNA from human cells. The first protocol is used to collect blood from patients/subjects/volunteers in tubes containing PAXgene™, a commercially available, proprietary mixture of reagents that, during blood draw, immediately lyses the cells and stabilizes mRNA, which can then be purified in the lab. The second protocol describes a method for extracting total RNA from harvested cells using TRI REAGENT® (Molecular Research Center, Inc.). For example, cultured cells can be processed and the extracted mRNA quantified to use as a positive control/standard dilution series. These standards are run alongside unknown test samples in order to allow for quantification. The standard set is used within a single assay. A third protocol describes the reverse transcription of extracted RNA and the use of the resulting cDNA in PCR amplification. A real-time PCR method is utilized to quantitate each enzyme as well as a housekeeping gene, which is used as a normalization factor in reporting enzyme expression levels.

(i) mRNA Purification from Blood Collected from Subjects in PAXgene™ Tubes

In order to analyze gene expression in individuals, blood may be collected in PAXgene™ tubes (PreAnalytiX GmbH), which contain a proprietary mix of additives that immediately lyses cells and protects mRNA. Blood samples can then be stored prior to laboratory processing in such a manner that accurately preserves the levels of mRNA found in peripheral blood mononuclear cells (PBMCs) in vivo. After blood collection in PAXgene™ tubes, total RNA is purified using the PreAnalytiX Blood RNA Kit following manufacturer's instructions. RNA amounts may be quantified and further processed immediately, or may be stored at 4° C. for several days. For longer periods of time, RNA samples are stored at −20° C. For quantification of eluted RNA, like samples are pooled together in one tube and mixed well. Total volume is measured and each sample is quantified using a spectrophotometer (A₂₆₀ reading). Where the concentration of RNA harvested is too dilute to perform RT-PCR, samples may be concentrated using a RNeasy MinElute Cleanup Kit (Qiagen GmbH).

(ii) RNA Extraction from Human Cells Using TRI REAGENT®

This protocol describes a method for extracting and purifying RNA from individuals' peripheral blood mononuclear cells (PBMCs) or cells harvested from tissue culture using TRI REAGENT® RNA/DNA/protein isolation reagent (Molecular Research Center, Inc.). For example, the human Jurkat E6-1 leukocyte cell line has been processed to generate a standard set. RNA purified by this method can also be tested as an unknown for DNA repair enzyme levels. For a standard set, the purified RNA can be quantitated in terms of each of the repair enzymes and the housekeeping gene (as the concentrations of each of these enzymes are not identical), serially diluted and then aliquoted, generating sets of 8 different dilutions. The standards are kept frozen until use. These Standard Sets can be run alongside test samples in order to perform quantitative RT-PCR.

Approximately 3×10⁶ human cells are pelleted by centrifugation (either cells from tissue culture or PBMCs harvested from human blood) and TRIREAGENT® (1 mL) is added to each tube of pelleted cells. Each tube is vortexed for 15 seconds. Chloroform (200 μl) is added to each sample tube containing the TRI REAGENT® followed by vortexing for 15 seconds. The samples are allowed to sit for 5 minutes before centrifuging at 12,000×g for 10 minutes at 16° C.

A portion of the aqueous phase from each sample tube (500 μl) is transferred to a tube containing a co-precipitate of glycogen. Pre-aliquoted 7.5 M Ammonium Acetate (2.6 ml) is added to 30 ml of pre-aliquoted 100% isopropanol. 500 μl of this mixture is added to each of the tubes that contain the aqueous phase of the extraction and co-precipitate. Tubes are vortexed and placed at 2-8° C. for at least 1 hour to precipitate the RNA.

Samples are centrifuged at 23,000×g for 15 minutes at 4° C. All but approximately 30 μL of the supernatant is removed. Ethanol (70%, 1 ml) is added to each tube and the samples are inverted to wash the pellet. All the supernatant is removed and the nucleic acid pellet is dried. 20 μL of a resuspension mix (3.3 mM DTT and 0.687 U/μL RNasin) is added to each sample, and the RNA is resuspended for 30 minutes at room temperature. RNA is quantified by spectrophotometry (A₂₆₀ reading). For a sample from which standards will be generated, the concentration of each of the repair enzymes is quantitated via Poisson limiting dilution dilution (Sykes et al. (1992) Biotechniques, 13(3):444-449). Briefly, the standard material is diluted such that when utilized in RT-PCR some of the reactions are positive and some are negative. Individual amplification reactions may contain zero, one, two, three or more copies of the target sequence. PCR cannot differentiate between samples that contain one or more copies of the target sequence (all show equally positive). However, samples that contain zero copies are negative and therefore can be used to estimate the number of target molecules per reaction. The data are interpreted using the following formula that assumes a generalized Poisson distribution for the probability of retrieving no copies of the target sequence per PCR:
λ=ln(1/p),

where p=[(the number of negative results)÷(the total number of reactions)] and λ equals the average number of copies of the sequence of interest per reaction. By dividing the λ value by the volume of diluted RNA tested and multiplying by the dilution factor, the starting concentration of the target DNA repair enzyme can be determined. Based on these starting concentrations, the stock is then serially diluted in prescribed increments (see e.g., table below) to give a series of standards of known concentration for each of the transcripts.

TABLE 2
Dilution scheme - DRE Standards with Jurkat E6-1 extracts

| | Stock |
|---|---|
| Standard | Fold Dilution |
| P8 | Undiluted |
| P7 | 10 |
| P6 | 100 |
| P5 | 1000 |
| P4 | 2000 |
| P3 | 10000 |
| P2 | 20000 |
| P1 | 100000 |

20 μL of each standard is aliquoted into tubes to give eight concentrations per standard set. Standard sets are stored at −70° C. Each standard tube contains enough RNA for a single reverse transcription reaction.

Reverse Transcription of mRNA and RT-PCR for DNA Repair Enzyme Quantification

In this procedure, RNA previously extracted from human cells is reverse-transcribed and PCR-amplified to quantitate DNA repair enzyme-specific nucleotide sequences. DNA repair enzymes that may be amplified include, but are not limited to of 8-oxoguanine DNA glycosylase (OGG1), MutY homolog (hMYH), MutT Homolog-1 (MTH1), Heme oxygenase 1 (HOX1), NEIL endonuclease VIII-like 1 protein (NEIL1 protein), Nth homolog 1 (NTH1 protein), excision repair cross-complementing protein (ERCC1), AP endonuclease (Ape-1), or superoxide dismutase (SOD-1). Amplification of mRNA by PCR may be by standard reverse transcription-PCR (RT-PCR) or using real-time RT-PCR

Real-Time PCR is the simultaneous thermocycling of PCR samples and measurement of template amplification through the use of fluorescent probe(s) designed to anneal to template and signal the degree of amplification occurring. For example, a TaqMan Probe may be used to quantify the PCR product. As used herein, a TaqMan Probe is a dual-labeled oligonucleotide probe that is designed to anneal to a target sequence between two oligonucleotide primers used for PCR amplification of a particular nucleic acid. The labels consist of a 5′ fluorescent dye that can be detected by a photohybrid within the real-time PCR instrument and a 3′ quenching dye that operates by fluorescence resonance energy transfer (FRET). FRET refers to a distance-dependent interaction between the electronic excited states of two dye molecules in which excitation is transferred from a donor molecule to an acceptor molecule. During template replication, the bound TaqMan probe can be degraded by Taq polymerase via the exonuclease activity present in the enzyme. This separates the fluorescent dye from the quencher, releasing a detectable fluorescence, signaling that amplification is occurring. In some cases, a FastStart Tag polymerase may be used. FastStart Taq is a Taq polymerase (Roche) with a chemical modification that blocks its activity at ambient temperatures. Upon preheating the reaction at 95° C., the polymerase becomes fully active, producing a “hot start” PCR.

For RT-PCR, up to 8 μg of each sample RNA in a volume of 36 μL is added to 6 μL of oligo(dT)₁₀ (150 ng/μl). For blanks, 36 μL of water is added to the appropriate tubes containing 6 μL oligo (dT)₁₀. 16 μL of each standard in a set along with 20 μL water is added to a tube containing 6 μL of oligo (dT)₁₀.

Samples containing RNA and oligo(dT)₁₀ are heated to 70° C. for a minimum of two minutes. The samples are then cooled to allow the oligo(dT)₁₀ to anneal to the mRNA. 30 μL of Reverse Transcription (RT) Mix (12 mM MgCl₂, 0.024 mg/mL BSA, 2.4×PCR buffer, 6.0 mM DTT, 3 mM dNTP, 7.13 U/μL Reverse Transcriptase) are added to each tube. Samples are mixed and then incubated at 37° C. for 40-80 minutes followed by a 90° C. incubation for two minutes. 150 μL of PCR Mix (2.73 mM MgCl₂, 0.07 mg/mL BSA, and 1.3×PCR buffer) is then added to each sample. 25 μL of each sample mixture is separately added to six fresh tubes. These different tubes are used to amplify the different repair enzymes. Some of these enzymes are multiplexed with each other and/or with the housekeeping gene UBC. The housekeeping gene is not limited to UBC; Beta-2-microglobulin, Beta-Actin, Glyceraldehyde-3-phosphatedehydrogenase (GADPH), and Hypoxanthine phosphoribosyl-transferase 1 (HPRT1) can also be used. Examples of DNA repair genes that may be amplified by real-time PCR, and primers and probe combinations are provided in Tables 3 and 4. Table 3 delineates genes that can be amplified in the same reaction (primer pairs are listed). Table 4 provides example primers and real-time PCR probes. Primer and probe sequences for specific repair enzymes are included. However, a variety of primers and probes specific these repair enzyme transcripts may be used. Or, other DREs may be assayed using appropriate primers and probes.

TABLE 3

| Multiplex Reactions | SINGLE REACTION |
|---|---|
| ERC1A/B + MTH 2A/B + UBC 2A/B | NEI 2A/B |
| APE 2A/B + SOD 2A/B + UBC 2A/B | |
| NTH 2A/B + HOX 1A/C + UBC 2A/B | |
| OGG 6A/B + UBC 2A/B | |
| MYH 6A/B + UBC 2A/B | |

To each of these six tubes having the primers as shown above, 10 μL of the appropriate primer/probe mix (1×PCR buffer, 0.04 mg/mL BSA, 1.36 μM-4.08 μM DNA repair enzyme primer pair(s), 0.27 μM-1.09 μM DNA repair enzyme TaqMan probe(s), 1.36 μM-2.72 μM UBC primer pair (if applicable), 0.35 μM-0.28 μM UBC TaqMan probe (if applicable), and 0.16 U/μL FastStart Taq polymerase) is added. The table below lists the primer and probe sequences for various repair enzymes. The table below lists the primer and probe sequences for various repair enzyme transcripts. The fluorescent dyes on the 5′-end of the probe are 6-carboxyfluorescein (6-FAM), 4,7,2′4′5′7′,-hexachloro-6-carboxyfluorescein (HEX), or N,N-(dipropyl)-tetramethylindodicarbocyanine (Cy5). The quenching moieties on the 3′ end of the probe is Black Hole Quencher-1 (BHQ-1). However, other fluorophores and quenchers may be used.

TABLE 4
Primer and Probe Sequences for Repair Enzyme RT-PCR.

| | | | SEQ ID |
|---|---|---|---|
| Enzyme | Primer | Sequence | NO: |
| Excision Repair | ERC 1A | 5′- TGTCCAGGTGGATGTGAAAGAT -3′ | 15 |
| Cross- | ERC 1B | 5′- AGGAGGTCCGCTGGTTTCT -3′ | 16 |
| Complementing | ERC TaqPr 1 | 5′- /6-FAM/CCAGCAGGCCCTCAAGG | 17 |
| Protein | | AGCT/BHQ-1/ -3′ | |
| MutT Homolog | MTH 2A | 5′- GGCTAGGAGGGAGCTGCA -3′ | 18 |
| | MTH 2B | 5′- TGGGCGCATTTCGTCG -3′ | 19 |
| | MTH TaqPr 2 | 5′- /Cy5/CGGTCTGACAGTGGA | 20 |
| | | CGCCCTG/BHQ-1/ -3′ | |
| Heme oxygenase | HOX 1A | 5′- GACGGCTTCAAGCTGGTGA -3′ | 21 |
| 1 | HOX 1C | 5′- TGCAGCTCTTCTGGGAAGTAGA -3′ | 22 |
| | HOX TaqPr 1 | 5′- /6-FAM/CCTCCCTGTACCACATCTAT | 23 |
| | | GTGGCC/BHQ-1/ -3′ | |
| 8-oxoguanine | OGG 6A | 5′- GACCAACAAGGAACTGGGAAAC -3′ | 24 |
| DNA glycosylase | OGG 6B | 5′- CTGAGCATGGCGGGATTG -3′ | 25 |
| | OGG TaqPr 6 | 5′- /6-FAM/CGCAGGTCGGCACTG | 26 |
| | | AACAGC/BHQ-1/ -3′ | |
| MutY homolog | MYH 6A | 5′- TGTCCGAGCCATTGGTGC -3′ | 27 |
| | MYH 6B | 5′- TGGCTGCTTGGTTGAAATCTC -3′ | 28 |
| | MYH TaqPr 6 | 5′- /6-FAM/CAGCAGCACCCTTGTTT | 29 |
| | | CCCAGC/BHQ-1/ -3′ | |
| Nth homolog | NTH 2A | 5′- CCTGACGGTGGACAGCATC -3′ | 30 |
| | NTH 2B | 5′- TATTTCACCTTGCTCCTCCAGA -3′ | 31 |
| | NTH TaqPr 2 | 5′- /Cy5/CCACGCTGGGCAAGCTC | 32 |
| | | ATCTAC/BHQ-1/ -3′ | |
| NEIL | NEI 2A | 5′- GGACAGAGTGGAGGACGCTTT -3′ | 33 |
| endonuclease | NEI 2B | 5′- GTCCTGCTGGAGGCTGGTC -3′ | 34 |
| VIII-like protein | NEI TaqPr 2 | 5′- /6-FAM/TTGCAGTCCTCTTAGGAAG | 35 |
| | | GTCTCTCTTTG/BHQ-1/ -3′ | |
| Superoxide | SOD 2A | 5′- CAAAGGATGAAGAGAGGCATGTT -3′ | 36 |
| Dismutase | SOD 2B | 5′- CATCTGCTTTTTCATGGACCAC -3′ | 37 |
| | SOD TaqPr 2 | 5′- /Cy5/CGGCCAATGATGCAATGG | 38 |
| | | TCTCC/BHQ-1/ -3′ | |
| AP endonuclease | APE 2A | 5′- ATTGGCTGGAGGGCAGATCT -3′ | 39 |
| | APE 2B | 5′- TTCTTGGCCTCTGGCTCTGT -3′ | 40 |
| | APE TaqPr 2 | 5′- /6-FAM/AGCTCATCCCCGTCTTC | 41 |
| | | CGCC/BHQ-1/ -3′ | |
| Ubiquitin-C | UBC 2A | 5′- GTTCCGTCGCAGCCGG -3′ | 42 |
| | UBC 2B | 5′- AGATCTGCATTGTCAAGTGACGAT -3′ | 43 |
| | UBC TaqPr 2 | 5′- /HEX/CAGCGATCCACAAACAA | 44 |
| | HEX | GAACCGC/BHQ-1/ -3′ | |

The level of expression of each of the DNA repair enzymes is determined via real-time PCR using the following conditions: 1 cycle 95° C. for 10 minutes; 45 cycles 60° C. for 1 minute followed by 95° C. for 2 seconds; 1 cycle at 40° C. for 30 seconds. Concentrations for each DNA repair enzyme and housekeeping gene are calculated by the real-time instrument software. Thus, a linear regression curve is made from known standard concentrations versus calculated cycle numbers (crossing points) (FIG. 4). Crossing points for unknown samples are applied to the linear regression curve to extrapolate concentrations for the unknowns. Analysis/quantitation may be performed entirely by the software, with no user input.

In most cases, housekeeping gene expression is simultaneously measured in multiplex PCR to provide a normalization factor for target enzyme loading concentration. In the case of NEI, which is amplified in non-multiplex fashion, the UBC value is obtained by averaging the results from all the other UBC results obtained for the same sample at the same time (in other multiplex reactions).

Repair enzyme concentration is reported as a ratio relative to the concentration of a housekeeping gene, for example, [DNA repair enzyme concentration÷housekeeping gene concentration]×10]. Resulting ratios are compared between test samples and normal reference ranges or standard curves generated from normal, age-matched results to indicate any up-regulation or down-regulation of expression.

### Example 4

### DNA Repair Capacity Analysis

This example provides methods by which an individual's capacity to repair damage induced in vitro on cells from blood samples can be used to make predictions and recommendations regarding their health status.

(i) DNA Repair Capacity Assay

The test can be performed by harvesting a patient's peripheral blood mononuclear cells (PBMC's) by collecting whole blood in Becton Dickinson Cell Prep Tubes as per the manufacturer's instructions. PBMC's are frozen for storage prior to assaying. Cells are frozen in a solution of 10% DMSO, 40% RPMI 1640 medium, 50% FBS and thawed in 80% RPMI 1640, 20% FBS, 40 μM dNTP's (10 μM each) or 10% Dextrose, 40% RPMI 1640, 50% FBS, 40 μM dNTP's with overnight incubation. For assaying, samples are split into three equal cell populations of 2×10⁵ cells each, one of which serves as a negative control, with no damage induced, and the other two in which DNA damage is induced (in this example, with 10 μM H₂O₂ for 10 minutes at 4° C.) (see FIG. 6). One of the cell populations in which DNA damage is induced serves as a positive control in that DNA damage is assayed without exposing cells to recovery conditions (cells are immediately mounted on slides during test sample recovery). The other serves as the test sample, wherein the cells are washed twice with cold phosphate-buffered saline (PBS) and centrifugation, then resuspended in RPMI 1640 containing 20% fetal bovine serum and 40 μM dNTP's and incubated in a 37° C., 5% CO₂ incubator for 1.0 hour to allow DNA damage repair. Prior to mounting on slides in low-melt agarose, Negative and Positive cells are washed twice with cold PBS. Test cells are washed once with cold PBS after recovery incubation. DNA damage is quantified in each of the 3 cell populations (Negative, Positive and Test) using the Comet Assay.

(ii) Quantifying DNA Degradation Using the Comet Assay

The Comet Assay capitalizes on the rapid quantification of DNA fragmentation associated with DNA damage. This assay, also referred to as a “Single Cell Gel Electrophoresis Assay,” is based on the alkaline lysis of labile DNA at sites of damage and on the loss of integrity of cell membranes. Additionally, DNA repair enzymes may be introduced into the assay to create AP sites that are transformed into strand breaks during alkaline treatment, thus increasing the amount of total damage to be visualized. The unwound and fragmented DNA is able to migrate out of the cell during electrophoresis and can be visualized using SYBR® Gold nucleic acid gel stain. Cells that have accumulated DNA damage appear as fluorescent comets with tails of DNA fragments. Undamaged DNA does not migrate far from the origin and normal cells appear round.

A commercial assay kit (e.g., Trevigen's CometAssay™ Kit) may be used. The assay is performed, according to kit instructions, with the more sensitive alkaline electrophoresis. Also, for some assays, an additional incubation step with the enzyme Endonuclease III is added in between cell lysis and alkaline unwinding of DNA to take advantage of the broad substrate specificity of the enzyme for mutated pyrimidine derivatives, so that any base adducts recognized by the enzyme will be converted to additional strand breaks. After slides have dried (post-electrophoresis), DNA in individual cells can be visualized under a fluorescence microscope and quantitated using image analysis software as per manufacturer's instructions. In this example, a 501 Nikon Eclipse microscope with a J-FL EPI-Fluorescence attachment, X-cite 120 power supply and lamp module, B-2A filter cube and Q-Imaging QI CAM camera is used alongside Andor Technology's Komet 5.5 software, with cells on slides stained with a 1/10,000-1/20,000 dilution of SYBR Gold nucleic acid staining dye. A cell image is viewed on the fluorescence microscope and on the associated monitor, selected by mouse click and the software automatically calculates the fluorescence levels in the head and tail and the tail length and moment, assigning a Comet score (in this case, the Olive Tail Moment measurement). The scores from 50 to 100 cells per sample are averaged. DNA repair is defined by the following equation: [1−(OTM_{Test}−OTM_{Neg}/OTM_{Pos}−OTM_{Neg})]×100. This is described as the % repair effected in the test sample during the recovery period. This % repair result can be directly compared to results from other cell samples and reference curves based on results from many other individuals.

### Example 5

### DNA Damage Susceptibility

As described above, DNA damage may be measured using a DNA Damage Susceptibility Assay. In general, the DNA Damage Susceptibility Assay works by measuring the activation of Poly(ADP-ribose) polymerase (PARP), a nuclear enzyme that contributes to DNA repair and is activated by strand breaks in DNA. Activated PARP hydrolyzes NAD(P)H into nicotinamide and ADP-ribose, and polymerizes the ADP-ribose onto nuclear proteins. The level or amount of damaged DNA may be directly proportional to the level of activated PARP and the related and proportional reduction of NAD(P)H levels (see, e.g., Nakamura et al. (2003) Nucl. Acids Res., 31:e104). Therefore, measurement of NAD(P)H levels can serve as a sensitive assay for the measurement of DNA strand breaks. A tetrazolium salt dye, XTT (2,3-bis(2-methoxy-4-nitro-5-sulfonyl)-2H tetrazolium-5-carboxanilide) may be used to measure relative levels of NAD(P)H; XTT is normally yellow in solution, but turns orange with reduction, which is facilitated through NAD(P)H (FIG. 7).

The DNA Damage Susceptibility Assay is a rapid, multiwell format, colorimetric test that quantitates DNA strand breakage in cell samples damaged in vitro compared to the damage level in negative controls of the same cell samples. Patient PBMC's are harvested, frozen and thawed in the same manner as described above in Example 4 (DNA RCA Assay). Cells are counted, pelleted and resuspended in PBS and aliquoted into two identical populations (labeled negative and positive), with enough cells in each for six replicates of 250,000 cells/100 μL/well in a 96-well plate. The positive population is exposed to a DNA-damaging agent, e.g., 500-1000 μM H₂O₂ for 20 minutes at room temperature. Both negatives and positives are then washed with PBS once; after centrifugation, pellets are resuspended to the same volume as before in dye-free, serum-free RPMI 1640 culture medium containing 36-58 units/mL catalase. Six 100-μL replicates of each negative and each positive from every sample are added to plate wells. In the assay, lowered levels of NAD(P)H are measurable by lowered XTT dye reduction such that there is less color change (yellow to orange). 100 μL of the dye-free medium containing catalase is added to each of six wells on the same plate as a blank sample. To each well containing cells and/or medium, 50 μL of XTT dye preparation is added. This preparation is comprised of dye-free, serum-free RPMI 1640 culture medium containing 1.2 mg/mL of XTT dye and 33.6 μL/mL of 1-methoxy-5-methylphenazinium methylsulfate (1-methoxy PMS). The multiwell plate is incubated in a 37° C., 5% CO₂ incubator for 60-120 minutes and then spectrophotometric readings are taken at 450 nM (to measure the dye) and 650 nm (to correct for background absorbance of plate). The OD₆₅₀ is subtracted from the OD₄₅₀ for every well and then the average blank value (calculated from the 6 replicate results) is subtracted from every negative and positive reading to obtain “blanked” results. The amount of damage incurred in a sample is calculated by the following formula (1−(Avg OD_{Pos}−Avg OD_{Neg}))×100, with smaller result values referring to less damage incurred. Thus, in the presence of DNA breaks, PARP is activated and diminishes cellular NAD(P)H concentration. Since NAD(P)H is required for the color change of the added XTT dye, DNA damage is indicated by little color change (yellow->orange, as measured on the spectrophotometer). Conversely, with little damage, PARP is not greatly activated, and almost-normal NAD(P)H levels lead to a much greater XTT color change that more resembles that observed in the negative (undamaged) control.

### Example 6

### Standard Curves or Reference Ranges for DNA Damage and DNA Repair Analysis

DNA samples from normal healthy individuals of different ages, individuals of different ages who have experienced an environmental, physiological, or lifestyle variable change, and individuals of different ages with different diseases are analyzed using selected DNA Damage and/or DNA Repair assays. The results are used to compile standard curves or references ranges indicating the expected DNA damage or repair levels for individuals in each category based on measurements obtained for the selected assay. A non-graphical representation of this is a collection of reference ranges for different demographic groups. Clinical study results from a large number of healthy and unhealthy individuals will contribute to the definition of reference ranges for each assay. Subsequent test samples giving results within reference ranges are considered “normal” for a healthy individual at a particular age. Results falling outside of range will be flagged as abnormal (indicating either possible high DNA damage level or low DNA repair level).

### Example 7

### Measurement of DNA Damage DNA Repair, and DRE Activity in Subjects

Results have been obtained from a relatively small number of volunteers for some of the tests developed and described herein. As the number of individuals tested was approximately 20, the results obtained indicate an estimate of the results expected for normal, healthy people generated from large clinical trials. The result ranges listed below represent the total range of values obtained and are useful for assay development purposes, but not reference ranges. Demographic information was not collected for the volunteers, so disparate results cannot be correlated with lifestyle, specific health factors, etc. All volunteers are generally healthy, 20-40 years of age (with a very small number ≧50 years). Some are smokers, but most are not. Numerous races and nationalities are represented in this small sampling.

Results for DRE Quantification are shown in the following Table 5.

TABLE 5

| Enzyme | Ratio Range Observed |
|---|---|
| ERCC-1 | 0.84-12.56 |
| MTH-1 | 1.03-16.97 |
| HOX-1 | 0.46-6.71 |
| NTH-1 | 0.14-2.57 |
| OGG-1 | 0.39-6.71 |
| MYH | 0.64-19.69 |
| APE-1 | 1.98-38.29 |
| SOD-1 | 3.95-62.5 |
| NEIL-1 | 1.07-8.47 |

Urinary 8-OH-dG levels were standardized with respect to creatnine. Urinary 8-OH-dG levels were found to be 1.74-6.68 ng/mg creatanine. The observed average was 4.47 ng/mg creatanine. For the DNA Damage Susceptibility Assay, the range observed for this group was 30-70% damage induced, with the average at 49%. Repair Capacity Analysis (using the Comet Assay) showed a range of 66.7-95.1%, with the average at 84.8%.

### Example 8

### Study of DNA Damage and Repair Activity in Tanning Salon Volunteers Study Design

This study will include no less than 250 but no more than 500 subjects. Subjects will be enrolled into the study and their urine and blood samples collected several days prior to, throughout the day of (for urine) or immediately after (for blood) acute UV-irradiation, and at least seven days after acute UV-irradiation. Subjects will be enrolled in the following groups: (1) Tanning bed volunteers/participants (individuals receiving at least one acute dose of UV-irradiation); and (2) Normal controls (individuals receiving no acute dose of UV-irradiation). The ratio of normal subjects to irradiation subjects will not be less than 1 to 10. All subjects will sign an IRB-approved Informed Consent Form prior to their enrollment and initial donation of a urine or blood sample under this protocol. Participants will be excluded if uncooperative or have potential exposure to other oxidizing agents (e.g., medications, UV-exposure, X-rays, radiation treatment).

Collection Schedule

Tanning participants will be asked to provide all urine voids for a 24-hour period several days prior to the day of tanning/UV-irradiation, and will repeat this collection on the day of tanning. An additional urine collection will be necessary no sooner than 7 days after the irradiation event.

Normal participants will be asked to provide all urine voids for a 24-hour period one day per week for at least three different weeks. Each voiding will be collected in a separate container. The participant will enter the time and date on the label of each container. Tanning participants will provide a blood sample before irradiation, immediately after and at least 7 days after irradiation. Normal participants will be asked to provide at least one blood sample sometime during the period of participation.

Assay Methods

Urine will be weighed to determine volume. An aliquot of each urine void will be analyzed for 8-hydroxy-2′-deoxyguanosine (8-OH-dG) by a quantitative high performance liquid chromatography with electrochemical detection (HPLC-ECD) assay and/or a liquid chromatography-mass spectrometry (LC-MS-MS) assay. A weight-averaged 24-hour urine “pooled sample” for each collection day for each subject will be prepared from aliquots of each collection. The pooled sample will also be analyzed for 8-OH-dG by HPLC-ECD and/or LC-MS-MS.

For measurement of DNA repair enzyme expression levels, blood samples can be collected in PreAnalytix PAXgene™ tubes. As discussed above, these tubes contain a proprietary solution that immediately lyses cells on contact and stabilizes mRNA molecules. This serves to ensure that no gene expression takes place in harvested cells after collection (due to a stress response, for example) and that any mRNA present in cells at collection is not degraded. This allows for an accurate measurement of in vivo enzyme expression levels free from collection artifacts. Stabilized mRNA of a number of DNA repair enzymes will be quantified by qRT-PCR.

For DNA repair capacity analysis, blood samples can be centrifuged immediately after collection in Vacutainer Cell Prep Tubes (CPT™) to separate the lymphocytes and monocytes (mononuclear leukocytes) in plasma from red blood cells and PMN (polymorphonuclear cells, such as neutrophils). PMN may release oxidative molecules into the plasma over time, damaging lymphocytes and monocytes if the cells are not centrifuged and separated in the tubes. Tubes should then be stored at 4° C. until delivery to the testing facility. Harvested mononuclear leukocytes will be challenged with a chemical DNA-damaging agent and degree of repair will be measured using the Comet Assay or another procedure that will similarly quantify DNA damage.

### Example 9

In a second clinical study, cancer patients undergoing treatment in the UCLA Radiation Oncology Department will donate urine and blood samples before, during, and several weeks after receiving physician-directed X-irradiation treatment. This study will serve to investigate the levels of excretion of 8-OH-dG in urine, and levels of DNA damage repair enzyme expression, DNA damage susceptibility and DNA repair capacity in blood cell samples from patients and also from normal control volunteers. These cancer patient and control results will be utilized in order to: (a) determine if there are average differences between cancer patients and normal controls; (b) investigate whether there are average differences for cancer patients of similar age, race, gender, etc. and/or disease diagnosis; (c) allow for correlation of an increase/decrease in any of these levels with ongoing cancer risk, since the selected cohort can be followed to assess further development of tumors; and/or (d) determine the effect, if any, radiation therapy has on oxidative damage and repair. The assay data and patient information (disease diagnosis, treatment and demographic data) collected from this clinical study will be correlated in order to better understand DNA damage clinically in individuals according to age and health status.

Although the foregoing invention has been described in some detail by way of illustration and example for purposes of clarity of understanding, certain changes and modifications may be practiced within the scope of the appended claims. All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed is:

1. A method to correlate the effect of at least one variable on at least one of DNA damage or DNA repair in an individual comprising the steps of:
(a) using a plurality of assays to measure at least one of DNA damage or DNA repair in the individual;
(b) determining whether the amount of at least one of DNA damage or DNA repair as measured in the individual differs from the amount of at least one of DNA damage or DNA repair for a plurality of control samples; and
(c) determining if there is a correlation between the levels of at least one of DNA damage or DNA repair in the individual to changes in the variable.

2. The method of claim 1, wherein the variable correlated to at least one of DNA damage or DNA repair comprises at least one of an environmental variable, a lifestyle variable, or a physiological variable.

3. The method of claim 1, wherein a positive correlation between an increase in DNA damage and the at least one variable provides an indication that the at least one variable may be associated with damage to the individual's DNA.

4. The method of claim 1, wherein a correlation between an increase in DNA repair and the at least one variable provides an indication that the at least one variable may be associated with damage to the individual's DNA.

5. The method of claim 1, wherein step (b) comprises measurement of both DNA repair and DNA damage by a plurality of assays for both DNA repair and DNA damage.

6. The method of claim 1, wherein an increase in the level of DNA damage that is not coupled with a comparable increase in the level of DNA repair, or a decrease in the level of DNA repair that is not coupled with a comparable decrease in the level of DNA damage, for the individual as compared to the plurality of controls, is indicative of a negative impact of the at least one variable on overall DNA damage accumulation in the individual.

7. The method of claim 1, wherein the control samples provide a range of reference values for the assay being used to monitor DNA damage or DNA repair.

8. The method of claim 1, wherein the control samples comprise samples taken from healthy subjects that are of a similar age as the tested individual.

9. The method of claim 1, wherein the control samples comprise samples taken from subjects that are exposed to the same variable or variables as the tested individual.

10. The method of claim 1, wherein the measurement of DNA damage is quantified by a DNA adduct assay.

11. The method of claim 10, wherein the DNA adduct assay comprises the measurement of DNA adducts using at least one of high performance liquid chromatography in combination with electrochemical detection (HPLC-ECD) or two-dimensional liquid chromatography in combination with tandem mass spectrometry (2D-LC-MS/MS).

12. The method of claim 10, wherein the DNA adduct assay measures the level of a DNA adduct comprising at least one of 8-hydroxydeoxyguanosine (8OHdG), 8-hydroxyguanosine (8OHG), 8-oxoguanine (8-oxo-G), 2,6-Diamino-4-hydroxy-5-formamidopyrimidine (FapyGua), 8-hydroxy-adenine/8-oxoadenine (8OHAde), O6-methyl-guanine, 4,6-Diamino-5-formamidopyrimidine (FapyAde), 5-hydroxy-cytosine (5-OH-Cyt), 5-Hydroxy-methylhydantoin (5-OH-5-MeHyd), 5-hydroxy-hydantoin (5-OH-Hyd), 2-oxoadenine (2-OH-Ade), or 5-Hydroxy-methyl-uracil (5-OH-Me-Ura).

13. The method of claim 1, wherein the measurement of DNA damage is quantified by a DNA break assay.

14. The method of claim 13, wherein the DNA break assay comprises at least one of a Comet Assay, an Aldehyde Reactive Probe Assay, or a DNA Ladder Assay.

15. The method of claim 1, wherein the measurement of DNA repair is quantified by at least one of a DNA Repair Enzyme assay, a Repair Capacity Analysis assay, or a DNA Damage Susceptibility assay.

16. The method of claim 15, wherein the DNA Repair Enzyme assay measures expression of at least one of 8-oxoguanine DNA glycosylase (OGG1), MutY homolog (hMYH), MutT Homolog-1 (MTH1), Heme oxygenase 1 (HOX1), NEIL endonuclease VIII-like 1 protein (NEIL1 protein), Nth homolog 1 (NTH1 protein), excision repair cross-complementing protein (ERCC1), AP endonuclease (Ape-1), or superoxide dismutase (SOD-1).

17. The method of claim 1, wherein the at least one variable being correlated to DNA damage or DNA repair comprises at least one of diet, physical activity, aging, pregnancy, stress, smoking, alcohol consumption, disease, disease treatment, drug treatment, antioxidant supplementation, cosmetic treatment, exposure to a carcinogen, or exposure to radiation.

18. The method of claim 17, wherein the cosmetic treatment is a chemical peel or laser resurfacing.

19. The method of claim 17, wherein the exposure to radiation involves exposure to X-ray radiation or ultraviolet radiation.

20. A method to correlate effect of at least one of DNA damage or DNA repair to an increased or a decreased risk for disease in an individual comprising the steps of:
(a) using a plurality of assays to measure at least one of DNA damage or DNA repair in the individual;
(b) comparing the level of at least one of DNA damage or DNA repair in the individual to the control samples;
(c) determining if the amount of at least one of DNA damage or DNA repair as measured in the individual differs from the amount of at least one of DNA damage or DNA repair for a plurality of control samples; and
(d) determining if there is a correlation between a change in at least one of DNA damage or DNA repair and a change in the risk of the disease for the individual.

21. The method of claim 20, wherein step (b) comprises measurement of both DNA repair and DNA damage by a plurality of assays for both DNA repair and DNA damage.

22. The method of claim 21, wherein an increase in the relative level of DNA damage coupled with a decrease in the relative level of DNA repair for the individual as compared to the plurality of controls is correlated with an increased risk of at least one disease that is associated with DNA damage.

23. The method of claim 21, wherein an decrease in the relative level of DNA damage coupled with a increase in the relative level of DNA repair for the individual as compared to the plurality of controls is correlated with an decrease risk of at least one disease that is associated with DNA damage.

24. The assay of claim 20, wherein the control samples provide a range of reference values for the assay being used to monitor DNA damage or DNA repair.

25. The assay of claim 20, wherein the disease comprises a disease of oxidative stress or DNA degradation.

26. The assay of claim 20, wherein the disease is cancer or a degenerative disease.

27. A method to correlate effect of at least one of DNA damage or DNA repair to an increased or a decreased risk for disease in an individual comprising the steps of:
(a) using a plurality of assays to measure at least one of DNA damage and DNA repair in the individual at an initial time;
(b) repeating the measurements of step (a) at one or more later time points measuring at least one of DNA damage and DNA repair in the individual at a later time;
(c) determining whether the amount of at least one of DNA damage or DNA repair as measured for at least one later time point differs from the amount of at least one of DNA damage or DNA repair at the initial time point; and
(d) determining if there is a correlation between a change in at least one of DNA damage or DNA repair and a change in the risk of the disease for the individual.

28. A kit to perform the method of claim 1.


SEQUENCE LISTING (44 sequences)

SEQ ID NO: 1 - DNA, 19 bp, Artificial Sequence
  Description of Artificial Sequence  Synthetic oligonucleotide primer BGL 1A derived from Homo sapiens
gagacgcatgagacgtgca

SEQ ID NO: 2 - DNA, 23 bp, Artificial Sequence
  Description of Artificial Sequence  Synthetic oligonucleotide primer BGL 1B derived from Homo sapiens
acacctctatccagcatcaactt

SEQ ID NO: 3 - DNA, 22 bp, Artificial Sequence
  Description of Artificial Sequence  Synthetic oligonucleotide primer BGL 1C derived from Homo sapiens
ggtcttgggtacaggagtttga

SEQ ID NO: 4 - DNA, 24 bp, Artificial Sequence
  Description of Artificial Sequence  Synthetic oligonucleotide primer BGL 4A derived from Homo sapiens
ttgtttgagacgcatgagacgtgc

SEQ ID NO: 5 - DNA, 30 bp, Artificial Sequence
  Description of Artificial Sequence  Synthetic oligonucleotide primer BGL 4B derived from Homo sapiens
aaatcttagaatgtgtttgtgagggaggaa

SEQ ID NO: 6 - DNA, 26 bp, Artificial Sequence
  Description of Artificial Sequence  Synthetic oligonucleotide primer BGL 4C derived from Homo sapiens
gttcccttgcttttctcttttcccat

SEQ ID NO: 7 - DNA, 22 bp, Artificial Sequence
  Description of Artificial Sequence  Synthetic oligonucleotide primer BGL 1F derived from Homo sapiens
cactggcttaggagttggactt

SEQ ID NO: 8 - DNA, 19 bp, Artificial Sequence
  Description of Artificial Sequence  Synthetic oligonucleotide primer MIT 1A derived from Homo sapiens
gtcccaccctcacacgatt

SEQ ID NO: 9 - DNA, 18 bp, Artificial Sequence
  Description of Artificial Sequence  Synthetic oligonucleotide primer MIT 1B derived from Homo sapiens
ttggcttagtgggcgaaa

SEQ ID NO: 10 - DNA, 20 bp, Artificial Sequence
  Description of Artificial Sequence  Synthetic oligonucleotide primer MIT 1D derived from Homo sapiens
ggtagatgtggcgggtttta

SEQ ID NO: 11 - DNA, 26 bp, Artificial Sequence
  Description of Artificial Sequence  Synthetic oligonucleotide primer MIT 4A derived from Homo sapiens
gtcccaccctcacacgattctttacc

SEQ ID NO: 12 - DNA, 26 bp, Artificial Sequence
  Description of Artificial Sequence  Synthetic oligonucleotide primer MIT 4B derived from Homo sapiens
tatgggagattattccgaagcctggt

SEQ ID NO: 13 - DNA, 22 bp, Artificial Sequence
  Description of Artificial Sequence  Synthetic oligonucleotide primer MIT 4C derived from Homo sapiens
ggtcggaggaaaaggttgggga

SEQ ID NO: 14 - DNA, 20 bp, Artificial Sequence
  Description of Artificial Sequence  Synthetic oligonucleotide primer MIT 1F derived from Homo sapiens
cgtgaaggtagcggatgatt

SEQ ID NO: 15 - DNA, 22 bp, Artificial Sequence
  Description of Artificial Sequence  Synthetic oligonucleotide primer ERC 1A derived from Homo sapiens
tgtccaggtggatgtgaaagat

SEQ ID NO: 16 - DNA, 19 bp, Artificial Sequence
  Description of Artificial Sequence  Synthetic oligonucleotide primer ERC 1B derived from Homo sapiens
aggaggtccgctggtttct

SEQ ID NO: 17 - DNA, 21 bp, Artificial Sequence
  Description of Artificial Sequence  Synthetic oligonucleotide primer ERC TaqPr 1 derived from Homo sapiens
  misc_feature (1)..(1): linked to 6-FAM
  misc_feature (21)..(21): linked to BHQ-1
ccagcaggccctcaaggagct

SEQ ID NO: 18 - DNA, 18 bp, Artificial Sequence
  Description of Artificial Sequence  Syntheti c oligonucleotide primer MTH 2A derived from Homo sapiens
ggctaggagggagctgca

SEQ ID NO: 19 - DNA, 16 bp, Artificial Sequence
  Description of Artificial Sequence  Synthetic oligonucleotide primer MTH 2B derived from Homo sapiens
tgggcgcatttcgtcg

SEQ ID NO: 20 - DNA, 22 bp, Artificial Sequence
  Description of Artificial Sequence  Synthetic oligonucleotide primer MTH TaqPr 2 derived from Homo sapiens
  misc_feature (1)..(1): linked to Cy5
  misc_feature (22)..(22): linked to BHQ-1
cggtctgacagtggacgccctg

SEQ ID NO: 21 - DNA, 19 bp, Artificial Sequence
  Description of Artificial Sequence  Syntheti c oligonucleotide primer HOX 1A derived from Homo sapiens
gacggcttcaagctggtga

SEQ ID NO: 22 - DNA, 22 bp, Artificial Sequence
  Description of Artificial Sequence  Synthetic oligonucleotide primer HOX 1C derived from Homo sapiens
tgcagctcttctgggaagtaga

SEQ ID NO: 23 - DNA, 26 bp, Artificial Sequence
  Description of Artificial Sequence  Synthetic oligonucleotide primer HOX TaqPr 1 derived from Homo sapiens
  misc_feature (1)..(1): linked to 6-FAM
  misc_feature (26)..(26): linked to BHQ-1
cctccctgtaccacatctatgtggcc

SEQ ID NO: 24 - DNA, 22 bp, Artificial Sequence
  Description of Artificial Sequence  Syntheti c oligonucleotide primer OGG 6A derived from Homo sapiens
gaccaacaaggaactgggaaac

SEQ ID NO: 25 - DNA, 18 bp, Artificial Sequence
  Description of Artificial Sequence  Synthetic oligonucleotide primer OGG 6B derived from Homo sapiens
ctgagcatggcgggattg

SEQ ID NO: 26 - DNA, 21 bp, Artificial Sequence
  Description of Artificial Sequence  Synthetic oligonucleotide primer OGG TaqPr 6 derived from Homo sapiens
  misc_feature (1)..(1): linked to 6-FAM
  misc_feature (21)..(21): linked to BHQ-1
cgcaggtcggcactgaacagc

SEQ ID NO: 27 - DNA, 18 bp, Artificial Sequence
  Description of Artificial Sequence  Syntheti c oligonucleotide primer MYH 6A derived from Homo sapiens
tgtccgagccattggtgc

SEQ ID NO: 28 - DNA, 21 bp, Artificial Sequence
  Description of Artificial Sequence  Synthetic oligonucleotide primer MYH 6B derived from Homo sapiens
tggctgcttggttgaaatctc

SEQ ID NO: 29 - DNA, 23 bp, Artificial Sequence
  Description of Artificial Sequence  Synthetic oligonucleotide primer MYH TaqPr 6 derived from Homo sapiens
  misc_feature (1)..(1): linked to 6-FAM
  misc_feature (23)..(23): linked to BHQ-1
cagcagcacccttgtttcccagc

SEQ ID NO: 30 - DNA, 19 bp, Artificial Sequence
  Description of Artificial Sequence  Syntheti c oligonucleotide primer NTH 2A derived from Homo sapiens
cctgacggtggacagcatc

SEQ ID NO: 31 - DNA, 22 bp, Artificial Sequence
  Description of Artificial Sequence  Synthetic oligonucleotide primer NTH 2B derived from Homo sapiens
tatttcaccttgctcctccaga

SEQ ID NO: 32 - DNA, 23 bp, Artificial Sequence
  Description of Artificial Sequence  Synthetic oligonucleotide primer NTH TaqPr 2 derived from Homo sapiens
  misc_feature (1)..(1): linked to Cy5
  misc_feature (23)..(23): linked to BHQ-1
ccacgctgggcaagctcatctac

SEQ ID NO: 33 - DNA, 21 bp, Artificial Sequence
  Description of Artificial Sequence  Syntheti c oligonucleotide primer NEI 2A derived from Homo sapiens
ggacagagtggaggacgcttt

SEQ ID NO: 34 - DNA, 19 bp, Artificial Sequence
  Description of Artificial Sequence  Synthetic oligonucleotide primer NEI 2B derived from Homo sapiens
gtcctgctggaggctggtc

SEQ ID NO: 35 - DNA, 30 bp, Artificial Sequence
  Description of Artificial Sequence  Synthetic oligonucleotide primer NEI TaqPr 2 derived from Homo sapiens
  misc_feature (1)..(1): linked to 6-FAM
  misc_feature (30)..(30): linked to BHQ-1
ttgcagtcctcttaggaaggtctctctttg

SEQ ID NO: 36 - DNA, 23 bp, Artificial Sequence
  Description of Artificial Sequence  Synthet ic oligonucleotide primer SOD 2A derived from Homo sapiens
caaaggatgaagagaggcatgtt

SEQ ID NO: 37 - DNA, 22 bp, Artificial Sequence
  Description of Artificial Sequence  Synthetic oligonucleotide primer SOD 2B derived from Homo sapiens
catctgctttttcatggaccac

SEQ ID NO: 38 - DNA, 23 bp, Artificial Sequence
  Description of Artificial Sequence  Synthetic oligonucleotide primer SOD TaqPr 2 derived from Homo sapiens
  misc_feature (1)..(1): linked to Cy5
  misc_feature (23)..(23): linked to BHQ-1
cggccaatgatgcaatggtctcc

SEQ ID NO: 39 - DNA, 20 bp, Artificial Sequence
  Description of Artificial Sequence  Syntheti c oligonucleotide primer APE 2A derived from Homo sapiens
attggctggagggcagatct

SEQ ID NO: 40 - DNA, 20 bp, Artificial Sequence
  Description of Artificial Sequence  Synthetic oligonucleotide primer APE 2B derived from Homo sapiens
ttcttggcctctggctctgt

SEQ ID NO: 41 - DNA, 21 bp, Artificial Sequence
  Description of Artificial Sequence  Synthetic oligonucleotide primer APE TaqPr 2 derived from Homo sapiens
  misc_feature (1)..(1): linked to 6-FAM
  misc_feature (21)..(21): linked to BHQ-1
agctcatccccgtcttccgcc

SEQ ID NO: 42 - DNA, 16 bp, Artificial Sequence
  Description of Artificial Sequence  Synthetic oligonucleotide primer UBC 2A derived from Homo sapiens
gttccgtcgcagccgg

SEQ ID NO: 43 - DNA, 24 bp, Artificial Sequence
  Description of Artificial Sequence  Synthetic oligonucleotide primer UBC 2B derived from Homo sapiens
agatctgcattgtcaagtgacgat

SEQ ID NO: 44 - DNA, 24 bp, Artificial Sequence
  Description of Artificial Sequence  Synthetic oligonucleotide primer UBC TaqPr 2 HEX derived from Homo sapiens
  misc_feature (1)..(1): linked to HEX
  misc_feature (24)..(24): linked to BHQ-1
cagcgatccacaaacaagaaccgc